(19) United States
(12) Patent Application Publication
(10) Pub. No.: US 2026/0265806 A1
(43) Pub. Date: Sep. 10, 2026

(54) COMPOSITIONS AND METHODS FOR DIAGNOSING AND TREATING AGE-RELATED NEURONAL CELL DAMAGE

(71) Applicant: THE REGENTS OF THE UNIVERSITY OF COLORADO, A BODY CORPORATE, Denver, CO (US)

(72) Inventors: Huntington POTTER, Denver, CO (US); Stefan SILLAU, Denver, CO (US); Christina M. COUGHLAN, Denver, CO (US); Mahiuddin AHMED, Denver, CO (US)

(21) Appl. No.: 19/543,124

(22) Filed: Feb. 18, 2026

Related U.S. Application Data
(63) Continuation of application No. PCT/US2024/043742, filed on Aug. 23, 2024.
(60) Provisional application No. 63/534,292, filed on Aug. 23, 2023.
(60) Provisional application No. 63/661,487, filed on Jun. 18, 2024.

(51) Int. Cl. C12Q 1/37; G01N 33/68

(52) U.S. Cl. CPC C12Q 1/37; G01N 33/6896; C12N 2310/14; G01N 2333/948; G01N 2800/2821; G01N 2800/52

(57) ABSTRACT

Embodiments of the instant disclosure relate to early diagnosis and intervention and/or treatment of neurodegenerative disorders and/or brain damage due to normal aging. Certain embodiments disclosed herein relate to quantifying concentrations of ubiquitin c-terminal hydrolase L1 (UCH-L1) in samples obtained from a subject to assess neuronal cell health and/or diagnose the subject with age-related neuronal cell damage. In some embodiments, a subject can be treated with an effective amount of a therapeutic to stabilize and/or reduce concentrations of UCH-L1 in the subject and treat the subject. In other embodiment, a targeted therapeutic agent, process, or task can be assessed for efficacy of reversing brain aging in a subject.

### PRIORITY

This application is a Continuation Application of PCT Application No. PCT/US2024/043742, filed Aug. 23, 2024, which claims priority to U.S. Provisional Application No. 63/534,292 filed Aug. 23, 2023; and U.S. Provisional Application No. 63/661,487 filed Jun. 18, 2024. These applications and all accompanying documents are incorporated herein by reference in their entireties for all purposes.

### GOVERNMENT FUNDING

These inventions were made with government support under Grant No. 1R01AG071151 from the National Institutes of Health (NIH) and Grant No. R01AI50305 from the National Institute of Allergy and Infectious Diseases (NIAID). The government has certain rights in these inventions.

### FIELD

Embodiments of the instant disclosure relate to detection and intervention and/or treatment of neuronal cell damage and in certain embodiments, age-related neuronal cell damage. Certain embodiments relate to neuronal regeneration in a subject in need thereof as indicated by therapeutic intervention and analysis of biomarkers related thereto.

### BACKGROUND

Neuronal cell damage is implicated in a variety of diseases and health conditions, including Alzheimer's Disease (AD) and age associated cognitive decline (AACD). Neuronal cell damage can be accelerated in certain subjects leading to increased propensity to decline in brain function and brain-related conditions. There is a need to identify new biomarkers associated with brain aging for early recognition of decline to allow interventive or early interventive treatment to reduce onset, treat, and/or reverse brain aging in a subject.

### SUMMARY

Embodiments of the instant disclosure relate to compositions and methods for diagnosing a subject with declining neuronal cell integrity and/or neuronal cell damage. In accordance with these embodiments, methods can include (a) measuring a concentration of ubiquitin C-terminal hydrolase L1 (UCH-LI) in a sample obtained from the subject; and (b) diagnosing the subject with declining neuronal cell integrity and/or neuronal cell damage if the concentration of UCH-L1 in the sample exceeds a concentration of UCH-L1 in a standard population of subjects. In certain embodiments, the standard population of subjects does not have declining neuronal cell integrity and/or increasing neuronal cell damage. In other embodiments, samples can be assessed for a concentration of UCH-L1 in a baseline sample from a subject and then a later sample obtained from the same subject to diagnose declining neuronal cell integrity and/or increasing neuronal cell damage in the subject. In accordance with these embodiment, the subject can include a subject where declining neuronal cell integrity and/or increasing neuronal cell damage has been identified in a related relative or multiple related relatives and the subject is monitored for declining neuronal cell integrity and/or increasing neuronal cell damage.

In some embodiments and further to paragraph [0005] above, the subject can be diagnosed with declining neuronal cell integrity and/or increasing neuronal cell damage if the concentration of UCH-L1 in the sample exceeds the concentration of UCH-L1 in the standard population by at least one, at least two, or at least three standard deviations of the standard population UCH-L1 concentrations. In some embodiments, severity of the declining neuronal cell integrity and/or increasing neuronal cell damage is proportional to a difference between UCH-L1 concentrations in the sample compared to the concentrations of UCH-L1 in the standard population of subjects.

In some embodiments and further to paragraphs [0005]-[0006] above, compositions, and methods for diagnosing a subject with declining neuronal cell integrity and/or increasing neuronal cell damage and/or astrogliosis/inflammation can further include measuring a concentration of at least one of neurofilament light chain protein (NfL) and glial fibrillary acidic protein (GFAP) in a sample obtained from the subject and in the standard population of subjects. In accordance with these embodiments, the subject can be diagnosed with declining neuronal cell integrity and/or increasing neuronal cell damage if the concentrations of NfL and/or GFAP in the subject exceed concentrations in the standard population of subjects such as a same aged population or younger population. In certain embodiments, the standard population of subjects does not have declining neuronal cell integrity and/or increasing neuronal cell damage. In other embodiments, samples can be assessed for a concentration of at least one of neurofilament light chain protein (NfL) and glial fibrillary acidic protein (GFAP) in a baseline sample from a subject and then a later sample obtained from the same subject to diagnose declining neuronal cell integrity and/or increasing neuronal cell damage in the subject. In accordance with these embodiment, the subject can include a subject where declining neuronal cell integrity and/or increasing neuronal cell damage has been identified in a related relative or multiple related relatives and the subject is monitored for declining neuronal cell integrity and/or increasing neuronal cell damage. In other embodiments, the subject is a healthy subject not having a related relative or multiple related relatives where declining neuronal cell integrity and/or increasing neuronal cell damage has been identified.

In certain embodiments and further to paragraphs [0005]-[0007] above, the sample obtained from the subject can include at least one of a blood and plasma sample. For example, in certain embodiments, the sample can include a plasma sample.

In other embodiments and further to paragraphs [0005]-[0008] above, the methods provided herein can further include administering a treatment to the subject to treat the declining neuronal cell integrity and/or increasing neuronal cell damage in the subject. Accordingly, in some embodiments, a method of treating declining neuronal cell integrity and/or increasing neuronal cell damage in a subject is provided. In certain embodiments, the methods include (a) measuring a concentration of UCH-L1 in a plasma sample obtained from the subject, (b) comparing the concentration of UCH-L1 in the plasma sample with a baseline concentration obtained from a standard population, and optionally, (c) administering a treatment for the declining neuronal cell integrity and/or increasing neuronal cell damage, performing a task or a process and preventing, reversing and/or treating declining neuronal cell integrity and/or increasing neuronal cell damage in the subject. In some embodiments, additional plasma samples can be obtained from the subject to assess progress of the condition or of the treatment for ameliorating the declining neuronal cell integrity and/or increasing neuronal cell damage in the subject.

In certain embodiments and further to paragraphs [0005]-[0009] above, the treatment is administered when the concentration of UCH-L1 in the plasma sample obtained from the subject exceeds baseline concentration from a plasma sample derived from a standard population as disclosed herein (e.g., standard age curve for UCH-L1, etc.). In accordance with these embodiments, the treatment can be administered to the subject when the concentration of UCH-L1 in the plasma sample obtained from the subject exceeds baseline concentration from a plasma sample of a standard population by at least one standard deviation or more. In some embodiments, baseline concentration from a plasma sample derived from standard population can be an average baseline concentration from the standard population. In accordance with these embodiments, a standard population can include subject of similar age, the same age or younger than the subject being tested.

In some embodiments and further to paragraphs [0005]-[0010] above, methods provided herein can further include (d) measuring a concentration of UCH-L1 in at least a second plasma sample obtained from the subject after administration of the treatment in (c) or after a predetermined period. In some embodiments, the treatment can be adjusted based on concentrations of UCH-L1 in the at least a second plasma sample measured in (d) compared to the concentrations of UCH-L1 in the plasma sample measured in (a).

In other embodiments and further to paragraphs [0005]-[0011] above, the treatment comprises administering an agent that decreases concentrations of UCH-L1 in the subject. In some embodiments, an agent that reduces concentrations of UCH-L1 in the subject can include granulocyte-macrophage colony-stimulating factor (GM-CSF) or similar agent. In certain embodiments, the GM-CSF is recombinant (e.g., sargramostim).

In some embodiments and further to paragraphs [0005]-[0012] above, the standard population can include an age-related standard population. In further embodiments, the average age of the standard population can be younger than the subject. For example, in certain embodiments, the standard population can include subjects less than 50 years old, less than 40 years old, or less than 30 years old. In certain embodiments, the standard population can include subjects between the ages of 5 and 20 or 5 and 30. In certain embodiments, the standard population can include a standard population of normal healthy subjects (e.g., not having a brain-related disorder).

In other embodiments and further to paragraphs [0005]-[0013] above, the methods herein can further include comparing the concentration of UCH-L1 in the subject to a concentration of UCH-L1 in another, different, standard population of normal subjects. For example, in some embodiments, the concentration of UCH-L1 in the subject can be compared to the concentration of UCH-L1 in a standard population at a similar age to the subject and to a concentration of UCH-L1 in a standard population at a younger age to the subject.

In other embodiments and further to paragraphs [0005]-[0014] above, methods of treating a subject for declining neuronal cell integrity and/or increasing neuronal cell damage and/or increasing glial inflammation is provided where the methods can include administering to the subject an agent that decreases plasma concentrations of UCH-L1 in the subject. In certain embodiments, the agent that reduces plasma concentrations of UCH-L1 in the subject can include granulocyte-macrophage colony-stimulating factor (GM-CSF). In certain embodiments, the GM-CSF is recombinant. For example, in some embodiments, an agent for treating a subject disclosed herein that directly or indirectly decreases plasma concentrations of UCH-L1 in the subject can include GM-CSF; for example, sargramostim.

In other embodiments and further to paragraphs [0005]-[0015] above, methods for testing efficacy of a potential therapeutic, process, or task for intervention (e.g., reversal) of declining neuronal cell integrity and/or increasing neuronal cell damage and/or increasing glial inflammation in a subject is provided. In some embodiments, the methods can include (a) measuring a concentration of UCH-L1 in a first sample obtained from a subject, (b) administering the potential therapeutic to the subject for a predetermined period, and (c) measuring a concentration of UCH-L1 in at least a second sample obtained from the subject after administration of the potential therapeutic, where efficacy of the potential therapeutic is determined by a reduction in concentration of UCH-L1 in the at least a second sample compared to the first sample. In other embodiments, the first and/or at least a second sample are each a plasma sample. In other embodiments, the methods for testing a therapeutic, process or task can include measuring other markers (e.g., neurofilament light chain protein (NfL) and/or Glial fibrillary acidic protein (GFAP)) in addition to UCH-L1 in the samples obtained from the treated subject. In other embodiments, a process or task can include at least one of administering an inhibitor of apoptosis or senolysis in neurons; an inhibitor of astrocyte activation that leads to lower GFAP expression or release; an inhibitor/antagonist of receptors on astrocytes for signaling molecules; an inhibitor of microglial activation that results in lower expression and/or release of activating molecules, monoclonal antibodies against interleukin-1 (IL-1) or interleukin-6 (IL-6), or other signaling molecules that activate astrocytes to make glial fibrillary acidic protein (GFAP), and combinations thereof. In yet other embodiments, an inhibitor can include an siRNA or a virus expressing siRNA targeted against a signaling molecule.

In some embodiments and further to paragraphs [0005] to [0016] above, the subject can be 45 years old or older, 55 years old or greater, or greater than 60 years, or greater than 70 years, or greater than 80 years, or greater than 90 years old. In certain embodiments, the subject can have or be at risk of developing a neurodegenerative disease (e.g., Alzheimer's Disease, Parkinson's Disease, Down syndrome, or Frontotemporal Dementia). In other embodiments, the subject can be a normal healthy subject. In certain embodiments, the subject is not at risk of developing and/or has not been diagnosed with a neurodegenerative disease (e.g., Alzheimer's Disease, Parkinson's Disease, Down syndrome, or Frontotemporal Dementia). In certain embodiments, the subject does not have any cognitive impairments. In other embodiments, the subject has not suffered from a traumatic brain injury. In still other embodiments, the subject does not have a chromosomal abnormality related to a brain condition or cognition. For example, in certain embodiments, the subject does not have Down syndrome. In some embodiments, the subject being analyzed is a female subject.

In other embodiments and further to paragraphs [0005]-[0017] above, compositions including detection agents for UCH-L1, NfL, and/or GFAP, and a plasma sample from a subject are contemplated. In certain embodiments, the plasma sample is obtained from a healthy subject not having a neurodegenerative disorder. In other embodiments, the plasma sample is obtained from a healthy subject having or developing a neurodegenerative disorder. In some embodiment, compositions disclosed herein include an antibody against each of UCH-L1, NfL, and GFAP.

In some embodiments and further to paragraphs [0005]-[0018] above, kits are contemplated herein for example for packaging, transport, and use. In other embodiments, kits can include instructions for obtaining samples from a subject and analyzing the samples for changes in expression and/or concentration of each of UCH-L1, NfL, and/or GFAP. In some embodiments, the samples are plasma samples.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIGS. 1A-1D represent exemplary plots indicating concentrations of ubiquitin C-terminal hydrolase-1 (UCH-L1) in subjects at different ages. Concentration of UCH-L1 at each time point is depicted as absolute values with point-wise standard errors in FIG. 1A, on a logarithmic scale with point wise standard errors in FIG. 1B or separated between males (lower three lines, formerly blue) and females (upper three lines, formerly red) both as absolute values (FIG. 1C) and on a logarithmic scale (FIG. 1D), according to certain aspects of the present disclosure.

FIG. 2 represents an exemplary plot indicating concentrations of UCH-L1 in plasma samples from healthy control participants (middle solid line with dotted line above and below solid middle line, formerly blue), mildly cognitively impaired individuals (upper three lines, formerly, green) and participants from a Phase II, trial with testing a therapeutic agent at baseline levels prior to treatment (diagonal steep solid line below and above control solid line, formerly red), according to certain aspects of the present disclosure.

FIG. 3 represents an exemplary plot indicating concentrations of UCH-L1 in plasma samples from healthy control participants (longer lines beginning at 0 years for age, black), in mild-to-moderate Alzheimer's Disease with treatment (short lowest lines, formerly blue) or without (upper short lines close to control data, formerly red) treatment with a pharmaceutical agent, according to certain aspects of the present disclosure.

FIGS. 4A-4E represent exemplary plots indicating concentrations of NfL in healthy subjects at different ages, plotted either as absolute values (FIG. 4A) or on a logarithmic scale (FIG. 4B) and concentrations of GFAP in healthy subjects at different ages plotted as absolute values (FIG. 4C), on a logarithmic scale (FIG. 4D) or separated between male (three lower lines, formerly blue) or female (three upper lines, formerly red) subjects (FIG. 4E) according to certain aspects of the present disclosure.

FIGS. 5A-5B represent exemplary plots indicating concentrations of NfL (FIG. 5A) or GFAP (FIG. 5B) in health controls (three lower lines, formerly blue) or in patients at baseline in a Phase II trial with recombinant human GM-CSF (three upper lines, formerly red), according to certain aspects of the present disclosure.

FIGS. 6A-6N represent exemplary images of immunohistochemical stains (FIG. 6A-6I) and quantification (FIG. 6J-6L) for Caspase-3 (lighter dots, formerly red) or DAPI (brighter dots, formerly blue) of the CA1 region (FIGS. 6A-6C and FIG. 6J), CA3 region (FIGS. 6D-6F and 6K) or the dentate gyrus (DG) region (FIGS. 6G-6I and 6L) of the hippocampus in age-matched F344 male WT rats (FIG. 6A, FIG. 6D, FIG. 6G), TgF2344-AD rats injected with saline at end of treatment (FIG. 6B, FIG. 6E, FIG. 6H), and TgF344-AD rats treated with GM-CSF at end of treatment (FIG. 6C, FIG. 6F, FIG. 6I) with FIG. 6J illustrates an exemplary bar graph of quantification of the representative images in FIG. 6A-6C, FIG. 6K illustrates an exemplary bar graph of quantification of images in FIG. 6D-6F, and FIG. 6L illustrates an exemplary bar graph of quantification of images in FIG. 6G-6I, according to certain aspects of the present disclosure. FIG. 6M illustrates exemplary images for staining of a first biomarker (light gray, green in the original image), a marker for healthy neurons and with 4′,6-diamidino-2-phenylindole) (DAPI) ((dark spots of darker gray, blue in the original image), a nuclear counterstain blue-fluorescence dye, FIG. 6M) for WT, far left panel; AD: Saline, middle panel and AD-GM-CSF, right panel of FIG. 6M. 6N represents a quantitative analyses, the percent area that stained positive for the biomarker in all three conditions (FIG. 6N), according to certain aspects of the present disclosure.

FIGS. 7A-7C illustrate some exemplary plots of concentrations of three biomarkers assayed in plasma samples from patients (e.g., Down syndrome, trisomy 21; DS; upper three lines, formerly red) compared to healthy controls (lower 3 lines, formerly blue, HC). These plasma biomarkers of neuronal death (UCH-L1; 7A) and damage (NfL; 7B) are higher at all ages of these patients and rise exponentially faster than healthy controls, starting about age 25 and 15 respectively for these markers. A biomarker of inflammation, FIG. 7C (e.g., GFAP) is the same level for Trisomy 21 Down syndrome and normal healthy controls at young ages and then rises exponentially faster starting about age 35 in DS patients compared to healthy controls, according to certain aspects of the present disclosure.

FIGS. 8A-8C illustrate bar graphs representing differences in biomarker levels measured in three different patient populations, healthy controls (HC), mild cognitive impairment (MCI) and Alzheimer's Disease (AD). Each of FIGS. 8A-8C illustrate levels of 3 different biomarkers measured in plasma samples obtained from these patients, according to certain aspects of the present disclosure.

FIG. 9 is a table overview of data related to FIGS. 8A-8C above, representing area under the curve of ROC analysis of a combined biomarker concentration to distinguish patients with normal cognition (Healthy controls; HC) compared to patients diagnosed with either MCI or AD, according to certain aspects of the present disclosure.

### DEFINITIONS

Terms, unless defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals.

As used herein, “treat,” “treating” or “treatment” can refer to treating, reversing, ameliorating, or inhibiting onset or inhibiting progression of a health condition or disease or a symptom of the health condition or disease.

### DETAILED DESCRIPTION OF THE INVENTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

In accordance with embodiments disclosed herein, markers disclosed herein can be used to assess and/or monitor declining neuronal cell integrity and/or increasing neuronal cell damage (including age-related neuronal decline or damage) and/or increasing glial inflammation. In accordance with these embodiments, assessing concentration of targeted markers disclosed herein in a sample from a subject can allow more efficient and accurate diagnosis of neuronal changes and age-related neuronal changes (e.g., age-related decline, cognitive decline, or neurodegenerative disease). Current imaging tools such as PET and Cerebrospinal fluid (CSF) analysis are for example, time-consuming, located in specified regions, expensive, invasive and can dramatically increase health disparity in certain populations and regions given these tools are not amenable or accessible to all communities or subjects. In certain embodiments, efficacy of interventions will be greatly enhanced if simple, rapid, and early intervention are possible as provided herein. Further, compositions and methods disclosed herein can be used to visualize neuronal aging in its earliest stages and over a lifetime of a subject. In certain embodiments, timing and frequency of interventions and efficacy of therapeutics can also be determined.

In some embodiments and further to paragraph [0035] above, biomarkers including, but not limited to, ubiquitin c-terminal hydrolase 1 (UCH-1), neurofilament light chain protein (NfL) and/or Glial fibrillary acidic protein (GFAP) can be used to assess neuronal health state of a subject. In other embodiments, it has been discovered that concentrations of one or more of these markers (e.g., UCH-L1) can be modified by certain therapeutics, including pro-inflammatory cytokines, that can be administered to treat a neurodegenerative condition including accelerated brain aging. In certain embodiments, methods and compositions disclosed herein relate to neuronal changes in the hippocampus—a region of the brain responsible for consolidation and retaining of memories. The hippocampus is divided into three regions: CA1, CA2, and CA3. These regions form the trilaminar loop, which is the processing center of long-term memory. Information processed in the trilaminar loop arrives through the dentate gyrus (DG), making this region critical for memory having a neuronal architecture and function that deviates from other cortical areas. One of the major differences of the DG compared to other brain regions is the finding that it can generate new principal neurons that are continuously integrated into a fully functional neural circuit throughout life.

Embodiments of the instant disclosure and further to paragraphs [0035]-[0036] above, compositions and methods disclosed herein relate to assessing concentrations of novel biomarkers for monitoring neuronal changes in a subject. In accordance with these embodiments, methods for diagnosing declining neuronal cell integrity and/or increasing neuronal cell damage in a subject are provided. In accordance with these embodiments the method can include (a) measuring concentration of ubiquitin C-terminal hydrolase L1 (UCH-LI) in a sample obtained from the normal subject; and (b) diagnosing the subject with age-related increasing neuronal cell damage or neuronal damage if the concentration of UCH-L1 in the sample exceeds a concentration of UCH-L1 of a standard population of subjects by a predetermined concentration difference. In certain embodiments, methods disclosed herein further include comparing the concentration of UCH-L1 to concentration of UCH-L1 of another, different, age-related population of normal subjects. In some embodiments, the age-related population can be younger than the normal subject being tested. In certain embodiments, the concentration of UCH-L1 in the standard population of subjects is an average concentration in the standard population. In some embodiments, the standard population includes subjects that are 5 years to 20 years old; or 5 years to 25 years old; or 5 years to 30 years old or other range; or are a similar or same age to a subject being tested. In some embodiments, the methods disclosed herein can include comparing the concentration of UCH-L1 in the subject to a standard population of age-matched subjects and to another different standard population of younger subjects (e.g., those ages 5 to 20). In certain embodiments, the sample(s) for comparison include blood samples. In other embodiments, the sample(s) for comparison include plasma samples from the subject. It is understood herein that any method known in the art for harvesting a blood sample and/or plasma sample contemplated herein can be used to assess concentrations of biomarkers contemplated herein.

In certain embodiments and further to paragraphs [0035]-[0037] above, the subject contemplated herein can be diagnosed with declining neuronal cell integrity and/or increasing neuronal cell damage or age-related increasing neuronal cell damage if the concentration of UCH-L1 in the sample(s) exceeds the concentration of UCH-L1 in the age-related standard population by at least one half, at least one, at least one and a half, at least two, at least two and a half, or at least three or more, standard deviations from the concentration of UCH-L1 in the standard or average standard population of UCH-L1 concentration. In some embodiments, the subject is diagnosed with declining neuronal cell integrity and/or increasing neuronal cell damage or age-related neuronal cell damage if the concentration of UCH-L1 in the sample(s) exceeds the concentration of UCH-L1 in the control population by at least one, at least one and a half, at least two, at least 2 and a half, at least three, at least three and a half, at least four, at least four and a half, at least five, at least five and a half, or at least six standard deviations of a standard population UCH-L1 concentration or average concentration thereof.

In some embodiments and further to paragraphs [0035]-[0038] above, methods disclosed herein can further include measuring a concentration of at least one additional marker of declining neuronal cell integrity and/or increasing neuronal cell damage or age-related increasing neuronal cell damage. In accordance with these embodiments, methods can further include measuring a concentration of at least one of neurofilament light chain protein 1 (NfL) and glial fibrillary acidic protein (GFAP) in the sample. In some embodiments, the sample is at least one of a blood sample and a plasma sample. In certain embodiments, the subject can be diagnosed with the increasing neuronal cell damage or age-related neuronal damage if concentrations of NfL and/or GFAP exceed concentrations in an age-related standard population of normal subjects (e.g., same aged or younger standard population or average thereof). In certain embodiments, the subject can be diagnosed with increasing neuronal cell damage or age-related neuronal cell damage if the concentration of at least one of NfL and GFAP in the sample exceeds the concentration of at least one of NfL and GFAP in the age-related population by at least one half, at least one, at least one and a half, at least two, at least two and a half or at least three or more standard deviations of the age-related population of at least one of NfL and GFAP concentrations. In certain embodiments, methods disclosed herein further include comparing the concentration of at least one of NfL and GFAP to another, different, age-related concentration of at least one of NfL and GFAP of normal subjects. In some embodiments, the age-related population can be younger than the normal subject being tested. In certain embodiments, samples from the subject can be obtained to assess concentration in a similar age and a younger age subject population.

In certain embodiments and further to paragraphs [0035]-[0039] above, the sample obtained from the subject can include a blood and/or plasma sample. In some embodiments, the sample can include a plasma sample. In other embodiments, the sample can include a cerebral spinal fluid (CSF) sample. In yet other embodiments, samples from a subject can include a subject having or suspected of developing a neurodegenerative disorder, or a healthy subject and can be obtained before, after and/or during a treatment regimen to stabilize and/or reverse neuronal damage in the subject. In some embodiments, the healthy subject can be experiencing early onset of neurodegeneration or accelerate neurodegeneration.

In some embodiments and further to paragraphs [0035]-[0040] above, the methods provided herein can further include administering a treatment to the subject to treat the declining neuronal cell integrity and/or increasing neuronal cell damage or age-related neuronal cell damage and stabilize and/or reverse progression and/or effects of increasing neuronal cell damage. Suitable treatments are described further below but can include any treatment, process or procedure known in the art to ameliorate or reverse declining neuronal cell integrity and/or increasing neuronal cell damage or age-related neuronal cell damage and stabilize and/or reverse progression and/or effects of increasing neuronal cell damage.

In certain embodiments and further to paragraphs [0035]-[0041] above, methods of treating declining neuronal cell integrity and/or increasing neuronal cell damage are provided. In certain embodiments, the methods of treating declining neuronal cell integrity and/or increasing neuronal cell damage can include (a) measuring a concentration of UCH-L1 and optionally, another biomarker contemplated herein in a sample (e.g., plasma) obtained from a subject, (b) comparing the concentration of UCH-L1 in the sample with a baseline concentration obtained from an standard population (e.g., an age-matched population or a younger population or average concentration thereof) and (c) administering a treatment for declining neuronal cell integrity and/or increasing neuronal cell damage; optionally, d) measuring concentration of UCH-L1 in at least a second sample (e.g., plasma) obtained from a subject after treatment; and optionally e) adjusting treatment intervention based on d). In certain embodiments, a treatment can be administered until the concentration of UCH-L1 in the at least a second sample in (d) is lower than the concentration of UCH-L1 in (a) and/or approaching the concentration of UCH-L1 in a selected standard population. In some embodiments, a treatment is administered until the concentration of UCH-L1 in the at least a second sample in (d) is equivalent to or less than a concentration of UCH-L1 in a standard population of subjects that are similar age, the same age or younger than the subject. In some embodiments, a treatment is administered until the concentration of UCH-L1 in (d) is equivalent or less than a concentration of UCH-L1 in a standard population of subjects that are about age of 5 to about 20 or other selected aged population. In accordance with these embodiments, one indicator of an effective treatment is lowering concentrations of one or more of UCH-L1, NfL, or GFAP or other signaling agent in the sample of the subject (e.g., plasma sample). In yet other embodiments, concentrations of one or more of UCH-L1, NfL, or GFAP or other signaling agent can be increased in the brain of the subject.

In certain embodiments and further to paragraphs [0035]-[0042] above, the treatment can be administered when the concentration of UCH-L1 in a plasma sample obtained from the tested subject exceeds a baseline concentration from a plasma sample obtained from a standard population or another pre-determined concentration. In further embodiments, methods are provided for treating increasing neuronal cell damage or age-related neuronal cell damage in a subject in need thereof. In certain embodiments, the method can include administering to a subject an agent that decreases concentration of UCH-L1 in the subject. In some embodiments, the method can include administering to a subject an agent that decreases concentration of UCH-L1 in the subject by a predetermined concentration, or to the same or different control age-related concentration. In some embodiments, the method can include administering to a subject an agent that decreases concentration of UCH-L1 in the subject to a concentration in a younger standard population (e.g., subjects that are about age of 5 to about 20) or a same-aged healthy standard population or average concentration thereof. In certain embodiments, treatment intervention can be short, over a course of a week to about one month. In other embodiments, treatment intervention can be prolonged, over one month, 3 months, 6 months, a year or more. In other embodiments, treatment intervention can be for the duration of the subject's lifetime or as determined by a health professional. In certain embodiments, more than one treatment can be administered; for example, if a first treatment fails to reduce UCH-L1 concentration in the plasma of the subject. In accordance with these embodiments, the second, or more, treatment(s) can be administered according to any of the embodiments described herein.

In some embodiments and further to paragraphs [0035]-[0043] above, a suitable treatment to be administered to the subject can include any agent that is effective at blocking, reversing, or otherwise minimizing neuronal damage caused by simply aging, a neuronal injury or disease or that blocks or reverses or minimizes adverse neuronal changes (e.g., age-related neuronal cell damage). In some embodiments, the agent can alter immune function (e.g., increase inflammation or stimulate the innate immune system). In other embodiments, the agent can decrease UCH-L1 concentration in the subject's plasma and/or increase UCH-L1 concentration in the subject's brain. It has surprisingly been found that certain pro-inflammatory innate immune system stimulating cytokines (e.g., granulocyte macrophage colony stimulating factors, GM-CSF) can, in fact, decrease UCH-L1 concentration in blood or plasma of subjects. In accordance with these embodiments, a treatment of a subject disclosed herein can include administering a therapeutically effective amount of granulocyte macrophage colony stimulating factor (GM-CSF) to the subject and reducing UCH-L1 concentration (optionally, other biomarkers contemplated herein) in a blood or plasma sample relative to a subject not treated with GM-CSF or to a UCH-L1 blood or plasma concentration in the subject before treatment.

GM-CSF is a monomeric glycosylated polypeptide signaling molecule which is typically secreted by immune cells such as macrophages, T cells, mast cells, natural killer (NK) cells, as well as normal tissue cells such as endothelial cells and fibroblasts. In the bone marrow, GM-CSF functions as a leukocyte growth factor, and stimulates hematopoietic progenitor cells to differentiate into monocytes and granulocytes. In addition to its growth factor function, GM-CSF also acts as an important modulator of immune responses. Upon stimulation, many types of immune cells produce and secrete GM-CSF where it can act both locally to enhance maturation and antigen presentation function of macrophages and dendritic cells, as well as in a paracrine fashion to recruit circulating neutrophils, monocytes, and lymphocytes to areas of infection and inflammation. Clinically, GM-CSF is used to encourage bone marrow and immune cell recovery in subjects who have undergone immuno-depleting treatments, for example, radiation therapy, chemotherapy, etc.

When described herein and contemplated for therapeutic treatment disclosed herein, GM-CSF can include any form of GM-CSF or biologically active fragment thereof or mimetic thereof (e.g., GM-CSF mimetic). In some embodiments, GM-CSF can include, but is not limited to, recombinant GM-CSF. In certain embodiments, the GM-CSF includes, but is not limited to, human recombinant GM-CSF. In other embodiments, GM-CSF can include human recombinant GM-CSF, including, but not limited to, sargramostim, molgramostim, or regramostim or other recombinant GM-CSF. In other embodiments, exogenous administration of a viral or plasmid vector or mRNA construct designed to encode GM-CSF can be administered and expressed in the subject. In some embodiments, the GM-CSF includes, but is not limited to other non-human mammalian recombinant GM-CSF (e.g., for a pet, a companion animal or livestock or other animal). In some embodiments, compositions including, but not limited to, GM-CSF can be administered to a subject diagnosed with a neurodegenerative condition. In other embodiments compositions including, but not limited to, GM-CSF can be administered to a subject at risk of an age-associated neuronal change. In some embodiments, compositions including, but not limited to, GM-CSF can be administered to an elderly subject (e.g., a subject older than 50 years, older than 60 years, older than 65 years, older than 70 years, older than 75 years, older than 80 years, older than 85 years, or older than 90 years old).

In some embodiments and further to paragraphs [0035]-[0046] above, compositions and methods herein can include administration of about 50 μg/m²/day to about 1000 μg/m²/day; about 100 μg/m²/day to about 750 μg/m²/day; about 150 μg/m²/day to about 600 μg/m²/day; about 200 μg/m²/day to about 500 μg/m²/day; about 200 μg/m²/day to about 400 μg/m²/day; or about 250 μg/m²/day of GM-CSF, recombinantly produced molecule thereof or fragment or analog thereof in a single treatment or multiple treatments per day. In other embodiments, a subject can be treated every other day, 2 times per week, once a week or other dosing regimen such as a periodic regimen. In some embodiments, compositions and methods herein can include administration of about 250 μg/m²/day of GM-CSF, recombinantly produced molecule thereof or fragment or analog thereof in a single dose or multiple doses. In some embodiments, two doses can be provided to the subject where the total concentration of GM-CSF or analog thereof is about 50 μg/m²/day to about 1000 μg/m²/day or about 100 μg/m²/day to about 500 μg/m²/day or about 200 μg/m²/day to about 300 μg/m²/day or about 250 μg/m²/day. In certain embodiments, recombinant GM-CSF can be sargramostim, molgramostim, or regramostim or other recombinant GM-CSF. In some embodiments, a recombinantly produced molecule or fragment thereof can be administered in a composition at a significantly lower concentration such as about 2.5 μg/m²/day to about 500 μg/m²/day of GM-CSF.

In other embodiments and further to paragraphs [0035]-[0047] above, compositions and methods can include a composition including, but not limited to, GM-CSF, a recombinantly produced molecule or fragment thereof, or an analog thereof formulated in a pharmaceutical composition, which can further include a pharmaceutically acceptable carrier or excipient. In certain embodiments, the GM-CSF is recombinantly produced and in certain embodiments, the recombinantly produced GM-CSF can be sargramostim, molgramostim, or regramostim or other recombinantly produced GM-CSF. In some embodiments, GM-CSF is delivered in a particle, such as a lipid nanoparticle or any other delivery system. In accordance with these embodiments, the pharmaceutical composition can be administered to the subject by any means known in the art. In other embodiments, the pharmaceutical composition can be administered to the subject by inhalation, subcutaneous, intravenous, intranasal, inhalation, intra-arterially, by slow-release microparticles or timed-released formulation, by targeted deposit directly to the central nervous system. In yet other embodiments, the pharmaceutical composition can be administered to the subject by inhalation, intranasal and/or subcutaneous administration. In some embodiments, the subject can be treated one time, two times, or three times daily for a predetermined period to reduce and/or prevent neuronal damage. In yet other embodiments, the pharmaceutical composition can be administered to the subject alone, in combined treatment regimens or in combination with other agents or treatments for treating, preventing, or ameliorating viral infections or viral infection-related conditions in the subject. In some embodiments, compositions and methods disclosed herein can be used in combination treatments to reduce onset, prevent, reduce progression of and/or treatment of neurodegeneration where other treatments can include any standard treatment for the condition. For example, it is contemplated to be used in combination with other neurodegenerative treatment agents which will be beneficial to improve outcome.

In yet other embodiments and further to paragraphs [0035]-[0048] above, compositions and methods disclosed herein can include (d) measuring concentration of UCH-L1 or other biomarker contemplated herein in at least a second sample obtained from the subject after administration of the treatment in step (c). In certain embodiments, the treatment in (c) can be adjusted based on the concentration of UCH-L1 measured in the at least a second sample in (d). For example, if the concentration of UCH-L1 in the second plasma sample is lower than the first plasma sample, the treatment can be reduced, or discontinued. If the concentration of UCH-L1 in the at least second sample is higher or unchanged from the first sample, the treatment can be maintained or increased. In certain embodiments, therefore, the concentration of UCH-L1 and optionally other biomarkers disclosed herein can be used as a marker of treatment efficacy in a subject. In other embodiments, measuring concentrations of UCH-L1 in a sample can be used to assess whether a given intervention in neuronal damage is effective for short and/or long-term treatment.

Accordingly and further to paragraphs [0035]-[0049] above, in other aspects of the present disclosure, methods of testing efficacy of a potential therapeutic for treating age-related neuronal cell damage in a subject is provided. In some embodiments, the method can include (a) measuring a concentration of UCH-L1 and optionally other biomarkers contemplated herein in a first sample obtained from a subject, (b) administering the target therapeutic to the subject, and (c) measuring a concentration of UCH-L1 in at least a second sample obtained from the subject after administration of the target therapeutic, where efficacy of the target therapeutic can be determined by a reduction in concentrations of UCH-L1 in the at least second sample compared to the first sample. In some embodiments, the first sample and/or at least the second sample can each include a plasma sample. In other embodiments, combined treatment regimens can be used and then samples from the subject obtained to measure concentration of UCH-L1 and/or other biomarkers to assess stabilization and/or reversal of neuronal damage as contemplated herein.

In some embodiments and further to paragraphs [0035]-[0050] above, treatment intervention of a subject experiencing neuronal damage can include any pro-survival and/or anti-apoptotic agent that specifically targets and/or reduces cell death; for example, neuronal cell death. In some embodiments, treatments disclosed herein can include GM-CSF or variants thereof. Other known treatments are contemplated herein as well assessing new treatments for effects by obtaining blood or plasma samples from a subject and measuring concentrations of biomarkers and biomarker combinations contemplated herein where reduction in the targeted biomarker in the sample indicates improved neuronal cell integrity in the subject. In other embodiments, a process or task contemplated herein can include at least one of administering an inhibitor of apoptosis or senolysis in neurons; an inhibitor of astrocyte activation that leads to lower GFAP expression or release; an inhibitor/antagonist of receptors on astrocytes for signaling molecules; an inhibitor of microglial activation that results in lower expression and/or release of activating molecules, monoclonal antibodies against interleukin-1 (IL-1) or interleukin-6 (IL-6), or other signaling molecules that activate astrocytes to make glial fibrillary acidic protein (GFAP), and combinations thereof. In yet other embodiments, an inhibitor can include an siRNA or a virus expressing siRNA targeted against a signaling molecule. In accordance with these embodiment, because GFAP measured in the plasma is an indication of astrogliosis and inflammation in the brain, which is an accelerator of brain aging and neurodegeneration, the findings here indicate that blocking an increase in expression of activators of astrogliosis, for example, IL-1 and IL-6, can be a treatment for aging and neurodegenerative diseases whose incidence or presence is increased with aging. In certain embodiments, blocking receptors of these activators, such as using an antibody or a small molecule or other receptor binding agent and/or blocking expression of these receptors by a small interfering RNA (SiRNA) either directly or as expressed by a virus such as AAV can be used as a therapeutic treatment against normal aging or age-related brain disease.

In some embodiments and further to paragraphs [0035]-[0051] above, subjects contemplated herein can be a human (e.g., adult, adolescent, child, infant or fetus) or non-human animal such as a pet (e.g., dog, cat, pig, rabbit) or companion animal (e.g., a horse), livestock or other animal. In certain embodiments, the subject has been diagnosed with a declining neuronal condition (e.g., including loss of neurons). In certain embodiments, the subject is suspected of having or developing a decline in neuronal function. In certain embodiments, the subject has been diagnosed as having or is suspected of developing neuronal decline as a result of aging. In some embodiments, the subject is elderly. In certain embodiments, the subject is 50 years old or greater, 55 years old or greater, 60 years old or greater, greater than 65 years old, greater than 70 years old, greater than 75 years old, greater than 80 years old, greater than 85 years old, or greater than 90 years old.

In certain embodiments and further to paragraphs [0035]-[0052] above, the age-related standard population of normal subjects, as used herein, can include individuals at the same age as the primary subject (e.g., the subject to be analyzed for marker concentrations). In accordance with these embodiments, the standard population of normal subjects can be “age-matched.’ In some embodiments, an age-related standard population of normal subjects can include individuals at a younger age than the subject (e.g., the subject to be analyzed for marker concentrations). Accordingly, in some embodiments, concentrations of UCH-L1 and other biomarkers disclosed herein (e.g., NfL and GFAP) in a normal subject can be compared to concentrations of UCH-L1 in an age-matched standard population of normal subjects (e.g., those at the same age). In some embodiments, concentrations of UCH-L1 in a normal subject can be compared to concentrations of UCH-L1 in a younger standard population of normal subjects. In some embodiments, when the standard population of normal subjects can be younger than the primary subject, the standard population of normal subjects may include subjects less than 50 years old, less than 40 years old, or less than 30 years old. For example, in one non-limiting embodiments, concentrations of UCH-L1 in a subject can be compared to the concentrations of UCH-L 1 in a standard population of normal subjects that are between 20 and 30 years old. In certain embodiments, samples for analysis and comparison can be plasma samples.

In certain embodiments and further to paragraphs [0035]-[0053] above, the tested subject can be at risk of developing a neurodegenerative disease (e.g., Alzheimer's Disease, Parkinson's Disease, or frontotemporal dementia, Down Syndrome (DS) or DS progression). In some embodiments, the subject is not at risk of developing a neurodegenerative disease (e.g., Alzheimer's Disease, Parkinson's Disease, or frontotemporal dementia, Down Syndrome (DS) or DS progression).

In certain embodiments and further to paragraphs [0035]-[0054] above, the subject can be a normal subject. As used herein the term “normal” subject refers to a subject that does not have any known neurological or age-related neuronal conditions or abnormalities. In some embodiments, the subject is not at risk of developing or has not been diagnosed with a neurodegenerative disease. In certain embodiments, the neurodegenerative disease can include, but is not limited to, Alzheimer's Disease, Parkinson's Disease, Down syndrome, or frontotemporal dementia. Further, in certain embodiments the subject does not have any cognitive impairments. In certain embodiments, the subject has not suffered from a traumatic brain injury. In certain embodiments the subject is not diagnosed with Down syndrome. In certain embodiments, the subject does not have a chromosomal abnormality.

### Pharmaceutical Compositions

In certain embodiments and further to paragraphs [0035]-[0055] above, pharmaceutical compositions are contemplated. In accordance with these embodiments, pharmaceutical compositions can include GM-CSF. In some embodiments, pharmaceutical compositions herein can include GM-CSF and at least one pharmaceutically acceptable excipient or carrier. As used herein, the term “pharmaceutically acceptable” refers to those compounds, materials, compositions, and/or dosage forms which are, within the scope of sound medical judgment, suitable for use in contact with the tissues, organs, and/or bodily fluids of a subject without excessive toxicity, irritation, allergic response, or other problems or complications commensurate with a reasonable benefit/risk ratio. As used herein, the term “pharmaceutically acceptable carrier” can refer to solvents, dispersion media, coatings, antibacterial agents, antifungal agents, isotonic and absorption delaying agents, or the like that are physiologically compatible. Pharmaceutically acceptable carriers suitable for use herein, include, but are not limited to, buffers that are well known in the art, and can be phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

In some embodiments and further to paragraphs [0035]-[0056] above, pharmaceutical compositions for use herein can be formulated for parenteral administration, such as intravenous, intracerebroventricular injection, intra-cisterna magna injection, intra-parenchymal injection, intra-renal, intradermal, subcutaneous, direct introduction to the brain or central nervous system (i.e., CSF) or a combination thereof. In some embodiments, pharmaceutical compositions for use herein can be formulated for local delivery to the brain or cerebral spinal fluid (CSF). In some embodiments, pharmaceutical compositions for use herein can be formulated for nasal delivery. In some embodiments, pharmaceutical compositions for use herein be formulated for parenteral administration can include pharmaceutically acceptable carriers including sterile liquids, such as water and oil, including those of petroleum, animal, vegetable, or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. In other embodiments, pharmaceutical compositions for use herein can further include additional agents, for example preservatives, buffers, tonicity agents, antioxidants and stabilizers, nonionic wetting or clarifying agents, viscosity-increasing agents, and the like. In some embodiments, pharmaceutical compositions described herein can be packaged in single unit dosages or in multi-dosage forms.

In some embodiments and further to paragraphs [0035]-[0057] above, formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. In accordance with some embodiments herein, aqueous solutions can be suitably buffered (preferably to a pH of from 3 to 9). The preparation of suitable parenteral formulations for use herein under sterile conditions can be readily accomplished by standard pharmaceutical techniques well known to those skilled in the art.

In some embodiments and further to paragraphs [0035]-[0058] above, pharmaceutical compositions herein can further include one or more pharmaceutically acceptable salts. Non-limiting examples of pharmaceutically acceptable salts include acid addition salts (formed from a free amino group of a polypeptide with an inorganic acid, or an organic acid. In some embodiments, the salt formed with the free carboxyl groups is derived from an inorganic base, or an organic base. In some embodiments, any of the pharmaceutical compositions herein can be used in therapeutic applications which are also disclosed herein.

In certain embodiments and further to paragraphs [0035]-[0059] above, kits are contemplated of use herein. In accordance with these embodiments, kits can include a composition disclosed herein containing at least one agent for detection of biomarkers disclosed herein and at least one container. In other embodiments, kits can include at least one composition including, but not limited to, GM-CSF or similar agent thereof or fragment thereof or mimetic thereof. In yet other embodiments, kits can include combinations of agents to treat or reduce age-related neuronal cell damage including, but not limited to, a GM-CSF-containing composition. In other embodiments, kits can include containers for storing one or more samples from a subject or a standard or average standard population sample as a control and/or for comparison of one or more biomarkers disclosed herein.

It is understood by those of skill in the art that an exponential increase in any product of a biological process implies the existence of at least one positive feedback loop that accelerates the process. Embodiments disclosed herein identified exponential rises in the cross-sectionally assessed markers of brain degeneration, for example, neuron loss and axon damage, as well as astrogliosis/neuroinflammation with age and strong correlations with each other implying the existence of such a positive feedback loop in the process of brain aging. In certain embodiments disclosed herein, rise in neuron loss and axon damage evident in plasma from early childhood is likely to induce gliosis/inflammation to phagocytose the resulting debris, which would initiate an ‘inflammaging’ cascade, resulting in a vicious cycle of more neuronal damage and death and more inflammation. It has been observed that a positive feedback loop involving neuronal loss/damage and inflammation in brain aging throughout life includes the same components as the already-established positive feedback loop that comes into play later, in the development of AD. It was discovered that gliosis/neuroinflammation in AD increases the expression of cytokines that lead through several steps to increased production of Aβ peptides and their polymerization into neurotoxic oligomers and filaments. Then, Aβ oligomers, in turn, further increase neuroinflammation through further activation of microglia and astrocytes. As disclosed herein, positive feedback loops can be an ideal target for developing inhibitors of pathogenic pathways. Further, this disclosure identifies that a long-approved immune stem cell stimulating drug as an example, GM-CSF/sargramostim and other marker reducing agents contemplated herein, can be repurposed, or directed to reverse age-associated neuronal loss in subject and illustrates a benefit of blocking a key step in a feedback loop of age-associated brain degeneration to stabilize and/or reverse these effects.

Practice of embodiments of the present invention employs, unless otherwise indicated, conventional techniques of molecular biology (including recombinant techniques), microbiology, cell biology, biochemistry, and immunology, which are well within the purview of the skilled artisan. Such techniques are explained fully in the literature.

Embodiments of the present invention is further illustrated by the following non-limiting examples, which are not to be construed in any way as imposing limitations upon the scope thereof. On the contrary, it is understood that other embodiments, modifications, and equivalents thereof which, after reading the description herein, may suggest themselves to those skilled in the art without departing from the spirit of the present invention or the scope of the appended claims.

### EXAMPLES

The following examples are included to illustrate certain embodiments. It should be appreciated by those of skill in the art that the techniques disclosed in the examples which follow represent techniques discovered to function well in the practice of the claimed methods, compositions, and apparatus. However, those of skill in the art should, in light of the present disclosure, appreciate that changes can be made in some embodiments which are disclosed and still obtain a like or similar result without departing from the spirit and scope of the invention.

### Example 1

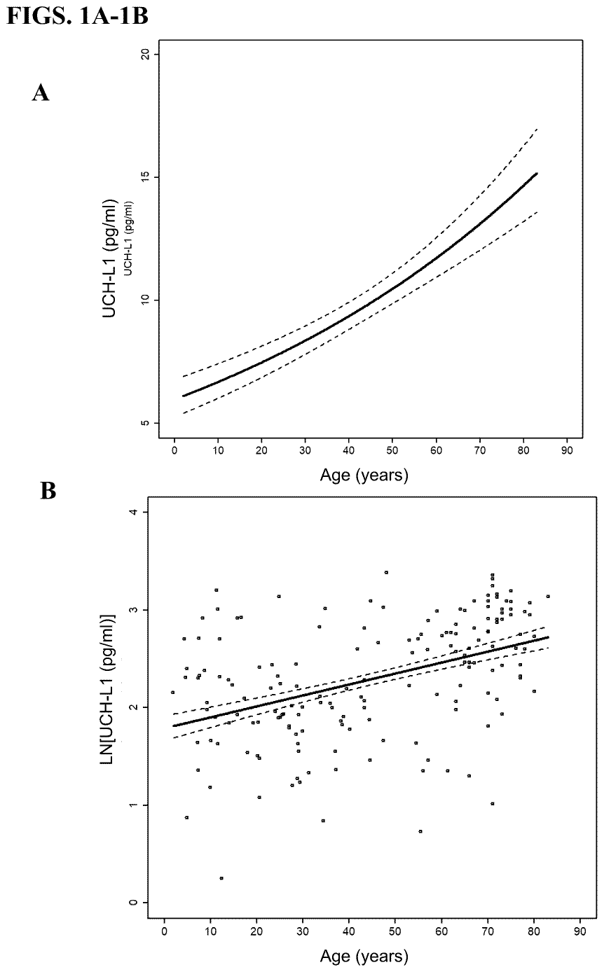

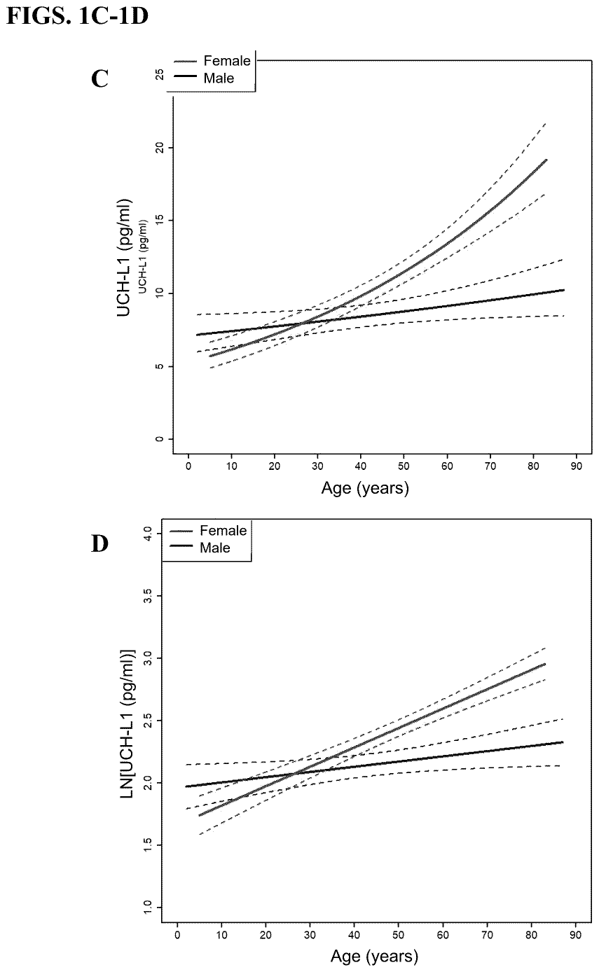

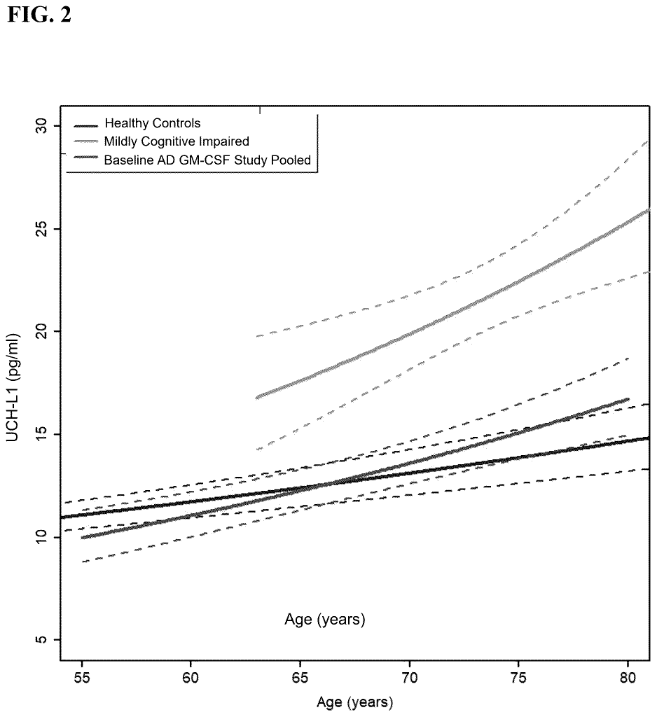

In one exemplary method, concentrations of UCH-L1 in plasma were evaluated in different populations. Specifically, UCH-L1 concentrations were evaluated in plasma samples from a large number (n=172) of healthy control participants between age 2 and 85, including 103 participants who served as healthy controls in the Human Trisome Project (HTP) of the Linda Crnic Institute for Down syndrome and 69 participants who served as healthy controls in the CUACC study of the role of inflammation in AD (termed Bio-AD), or the MS healthy controls biomarker study (n=145) (3 participants lacked usable UCH-L1 data). The assessments were determined using the Quanterix SIMOA® platform, which was also used to assess the plasma concentrations of UCH-L1 and other biomarkers in a previous Phase II, double-blind, randomized, placebo-controlled trial with recombinant human GM-CSF/sargramostim (the “sargramostim/GM-CSF AD trial”) in participants with mild-to-moderate Alzheimer's Disease (AD). It was found that plasma UCH-L1 concentrations increase exponentially with age across the entire age spectrum increasing from 6.212 pg/ml at age 2 to approximately 15.5618 pg/ml at age 85 (p<0.0001=5.504×10−8)) (FIG. 1A and FIG. 1B). Of particular interest is the finding that most of the age-associated increase in plasma concentrations of UCH-L1 occurs in females (FIG. 1C-FIG. 1D, estimated female change per year=1.448%, 95% CI: (0.942%, 1.957%), p=3.635×10⁸; estimated male change per year=0.582%, 95% CI: (−0.036%, 1.203%), p=0.0650; sex difference p=0.0342; estimate of the ratio of ratios=0.99146, 95% CI: (0.98362, 0.99936))female change per year=1.567%, p<0.0001; male change per year=0.419%, p=NS; sex difference p=0.0242). Therefore, FIG. 1A-1D demonstrate that concentrations of UCH-L1 increase as an individual ages and that this difference is more pronounced in females over males.

### Example 2

In another exemplary method, concentrations of UCH-L1 were measured in plasma samples obtained from 36 participants with mild-to-moderate Alzheimer's Disease (AD) from the sargramostim/GM-CSF AD trial taken at the baseline visit prior to treatment with sargramostim/GM-CSF or placebo. These data as well as data from 32 participants with mild cognitive impairment (MCI) due to AD from the Bio-AD study are plotted in FIG. 2 together with the data from the 317 healthy controls in FIG. 1. The correlations between absolute UCH-L1 concentrations and age and the point wise standard errors are illustrated. The results demonstrate that the plasma concentrations of UCH-L1 in the participants with MCI due to AD are higher overall than the plasma UCH-L1 concentrations of the healthy control participants at the mean age for the Leukine AD patients, 67.8 years (p=0.0088), whereas the plasma UCH-L1 concentrations of the mild-to-moderate AD participants are approximately the same as those of the healthy control participants. This suggests that during the progression of AD, UCH-L1 measures of neurodegeneration increase early during the MCI phase and then fall back towards normal, perhaps due to the loss of UCH-L1-releasing neurons.

### Example 3

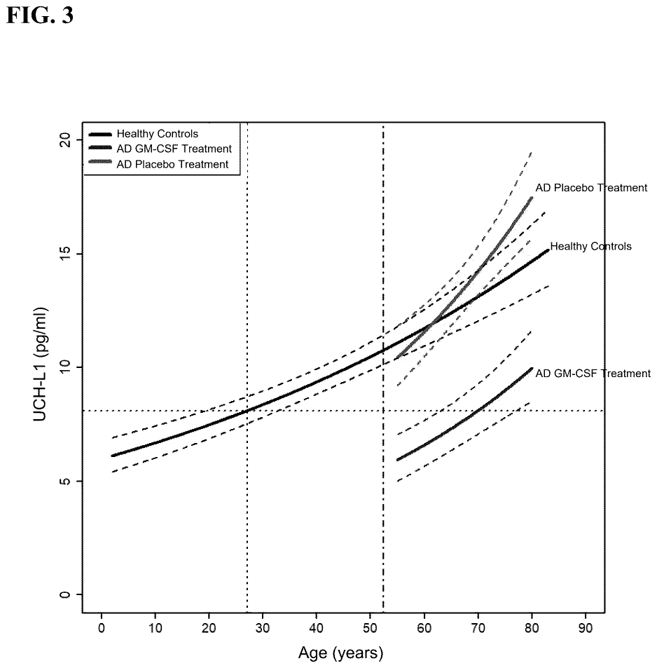

In another exemplary method, the concentrations of UCH-L1 in plasma of mild-to-moderate AD participants at the end of treatment with sargramostim/GM-CSF (250 mcg/m²/day subcutaneous injection, five days/week for three weeks) or placebo were plotted with the data from healthy control participants in FIG. 3. This shows that the age-associated exponential increase in plasma UCH-L1 concentrations remains, but the absolute values are greatly reduced after GM-CSF treatment (p=0.0008, at mean Leukine age of 67.8 years). Indeed, sargramostim/GM-CSF treatment reduces the concentrations of UCH-L1 in plasma of trial participants with mild-to-moderate AD to an average concentration equivalent to that found in healthy control participants many decades younger (FIG. 3). More specifically, a baseline calibrated, 67.8-year-old AD patient treated with sargramostim/GM-CSF was estimated to have a UCH-L1 concentration of 6.47 pg/ml (geometric mean, not baseline calibrated), which is equivalent to that of a 5.4-year-old healthy control participant. Comparing the baseline calibrated, 70-year-old sargramostim/GM-CSF-treated mild-to-moderate AD participant to the healthy control participant age curve, the differences would be statistically significant (two-sided alpha=0.05) from healthy control participants older than 37.8 years. The healthy control age equivalent of the plasma concentrations of UCH-L1 after GM-CSF treatment ranged from around 5 years old for 55-year-old AD participants to around 45 years old for 80-year-old AD participants. UCH-L1 concentrations in plasma from sargramostim/GM-CSF-treated mild-to-moderate AD trial participants at the end of treatment are statistically indistinguishable from those of healthy control participants below age 37.8.

### Example 4

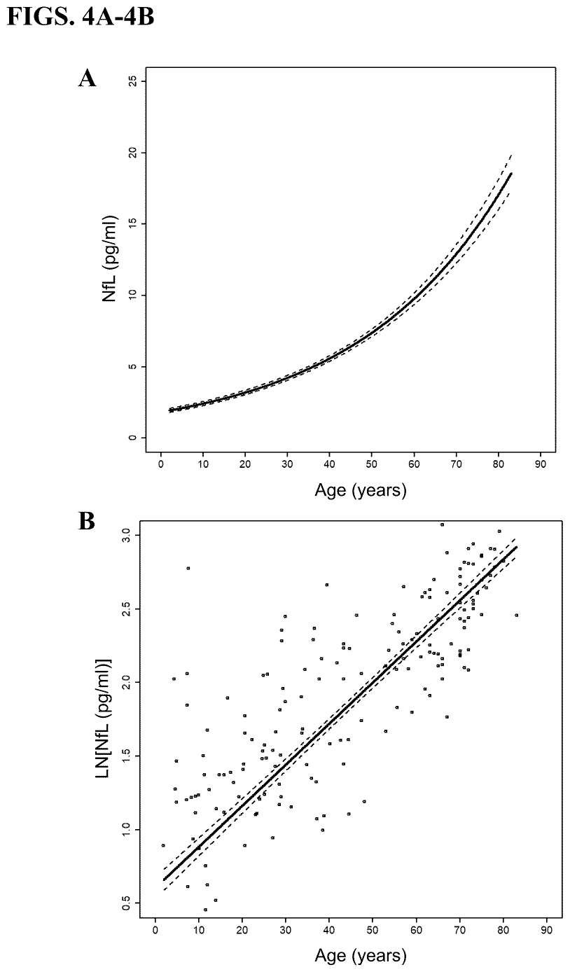

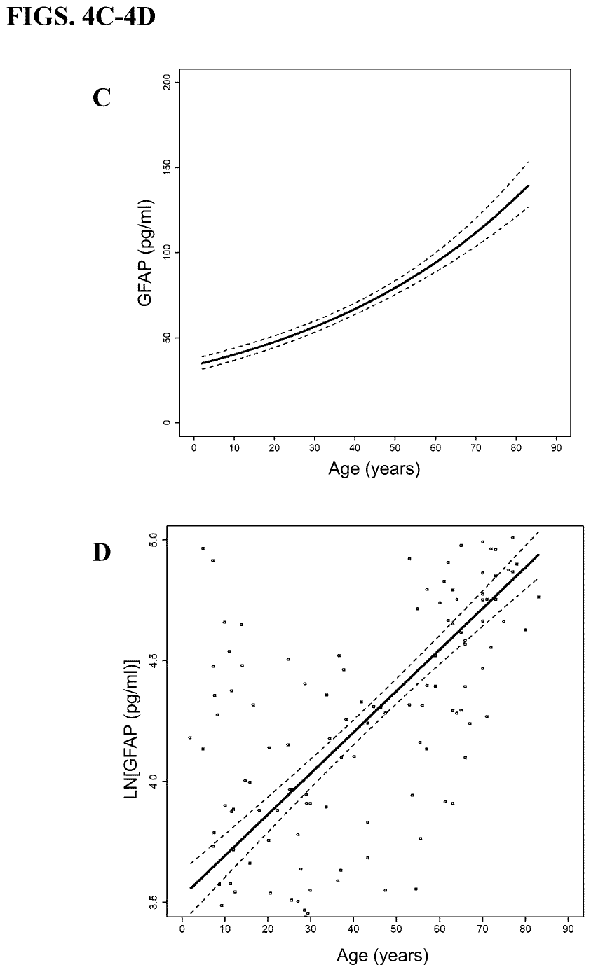

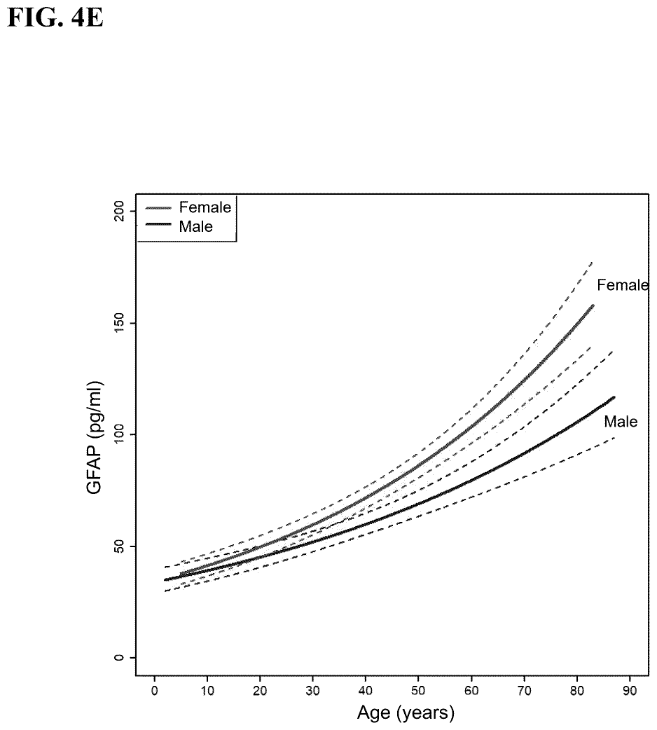

In another exemplary method, plasma concentrations of neurofilament light chain protein (NfL) as another measure of neurodegeneration and concentrations of Glial fibrillary acidic protein (GFAP) as a measure of astrogliosis, were measured. Specifically, in one exemplary method, the effect of age and sex on plasma concentrations of NfL and GFAP was determined in the 173 healthy control participants. As illustrated in FIG. 4, plasma concentrations of both NfL (FIGS. 4A and 4B) and GFAP (FIGS. 4C and 4D) also increased exponentially with increasing age in healthy control participants (p<0.0001), and the slopes of the log-transformed curves are greater than those observed for UCH-L1 with increasing age such that NfL plasma concentrations increase by 2.469% per year, 95% CI: (2.225%, 2.714%), p<2.220×10⁻¹⁶, GFAP plasma concentrations increase by 1.696%, 95% CI: (1.404%, 1.989%), p<2.220×10¹⁶. The effect of sex is also different for the GFAP plasma biomarker (FIG. 4E). Both females and males in our combined three cohorts that span ages 2-85 showed an apparent overall exponential age effect for plasma GFAP concentrations for both sexes (estimated female change per year=1.803%, 95% CI: (1.431%, 2.177%), p<2.220×10⁻¹⁶; estimated male change per year=1.496%, 95% CI: (1.037%, 1.957%), p=4.240×10⁻¹⁰, with no significant difference between males and females (FIG. 4E), in contrast to UCH-L1 where females exhibited a significantly greater change with age (e.g., see FIG. 1C and FIG. 1D).

### Example 5

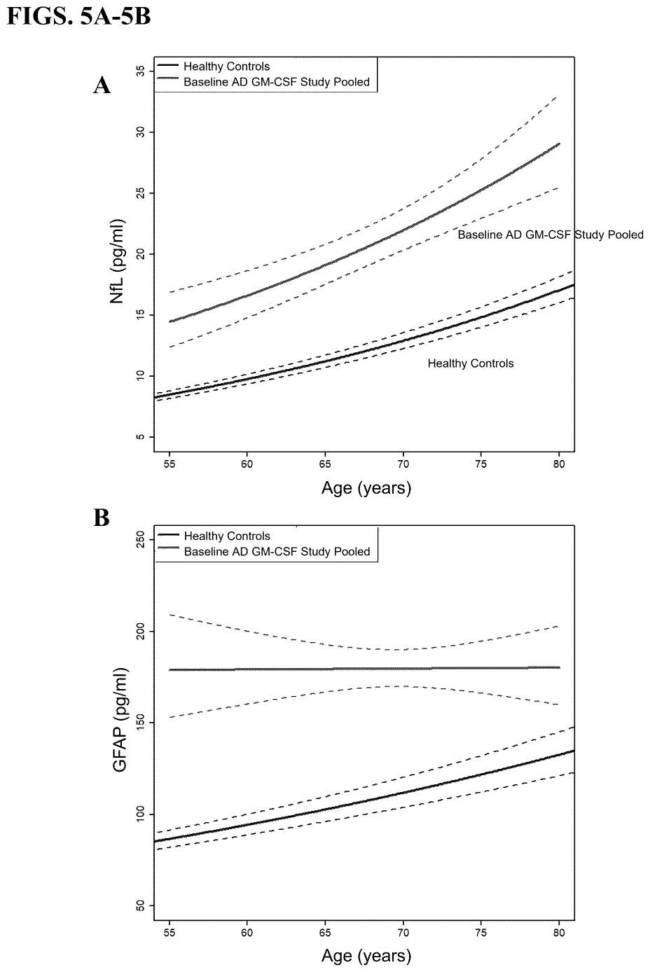

Although no effects of sargramostim/GM-CSF treatment were observed on NfL or GFAP plasma concentrations in participants with mild-to-moderate AD at the end of treatment, in another exemplary method, whether participants with mild-to-moderate AD showed age related differences in plasma NfL or GFAP concentrations compared to healthy control participants was determined. The concentrations of NfL and GFAP in plasma samples taken at the baseline visit of participants with mild-to-moderate AD in the sargramostim/GM-CSF trial prior to treatment with sargramostim/GM-CSF or saline were compared to the concentrations of these biomarkers in healthy control participants. As illustrated in FIGS. 5A-5B, a diagnosis of mild-to-moderate AD was associated with higher average concentrations of both NfL (FIG. 5A) and GFAP (FIG. 5B) compared to age-matched healthy control participants (both comparisons p<0.0001), as expected. Interestingly, the plasma concentration of NfL showed a clear age effect in the participants with mild-to-moderate AD, whereas GFAP did not.

### Example 6

In another exemplary method, the mechanism by which GM-CSF treatment reduces plasma concentrations of UCH-L1 was examined by using, in one exemplary method, aged TgF344-AD rats (18-20 months of age) which are a model of AD that shows the complete brain pathology of human AD (amyloid and Tau deposition and neurodegeneration).

The TgF344-AD rat was developed as a model of AD by inserting transgenes that express the Swedish mutant human APP (APPsw) and mutant human presenilin 1 (PSEN1 delta E9) genes that cause familial AD. As a result, and in part because the rat MAPT (Tau) gene resembles the human version, the rats overexpress human Aβ peptide and develop the full complement of human AD brain pathology: amyloid deposits, p-Tau positive neurofibrillary tangles, and neuronal loss. In one exemplary method, 20-month-old TgF344-rats were injected subcutaneously with GM-CSF (83.3 μg/kg/day; 5 days/week) or with saline (200 μl/day) for 24 injections total over 32 days. On day 32 brain tissues were processed for immunohistochemistry for Caspase-3 (a marker of apoptosis-induced neuronal damage that is increased in humans and animal models of AD and in aging) and stained for Caspase-3, a marker of apoptosis. Data from the treated TgF344-AD rats were also compared to age-matched wild-type control F344 rats.

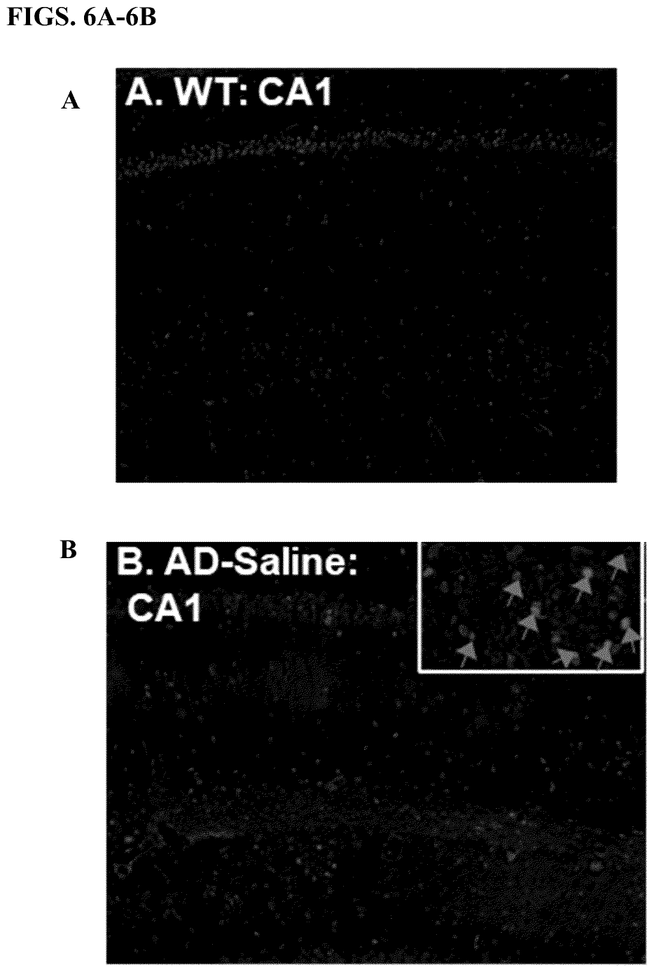

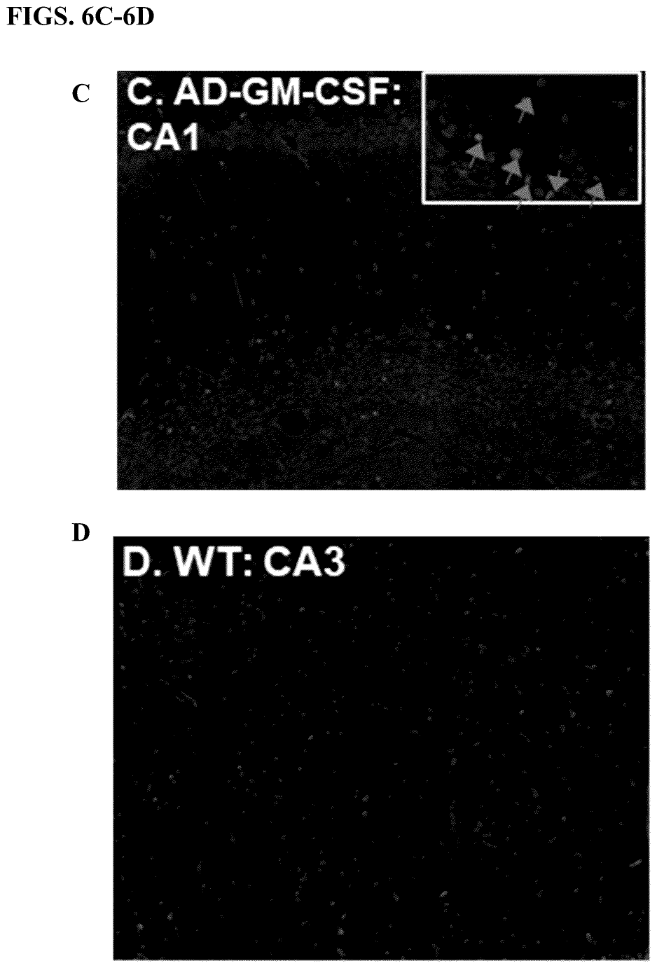

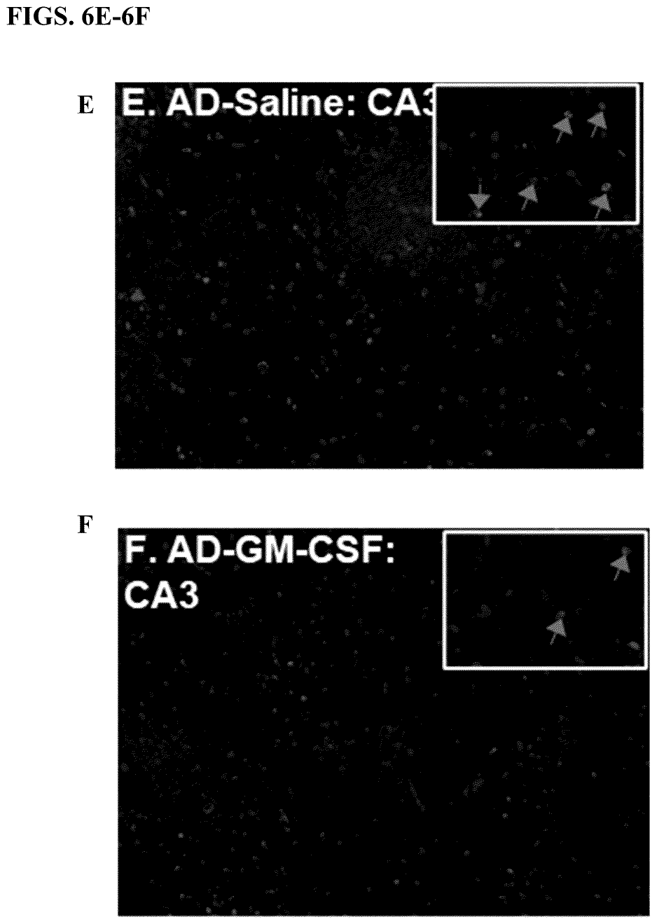

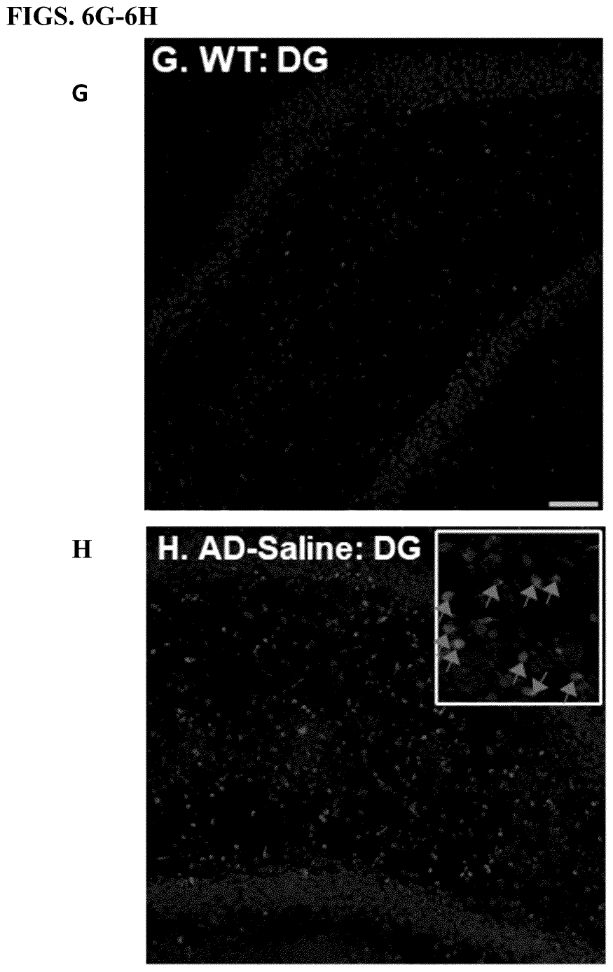

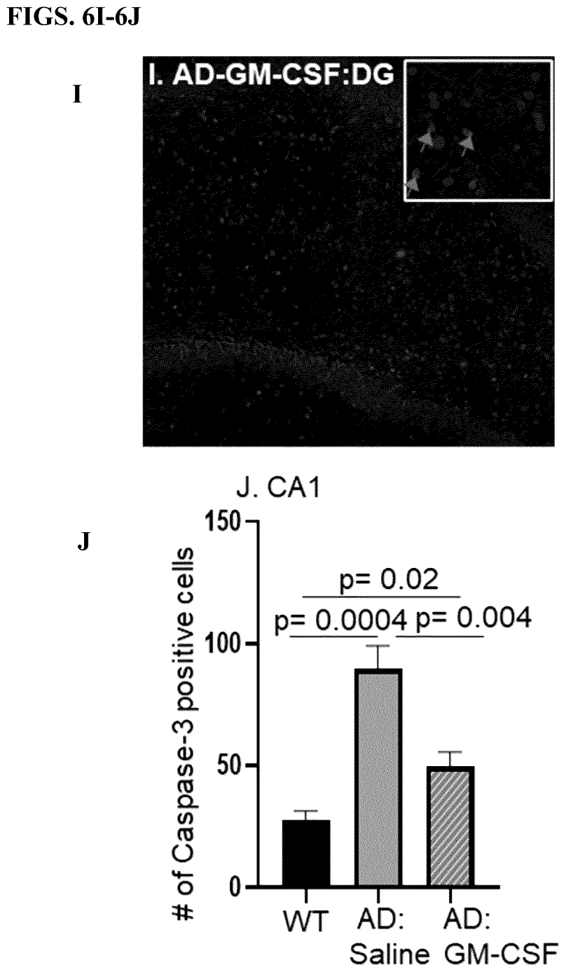

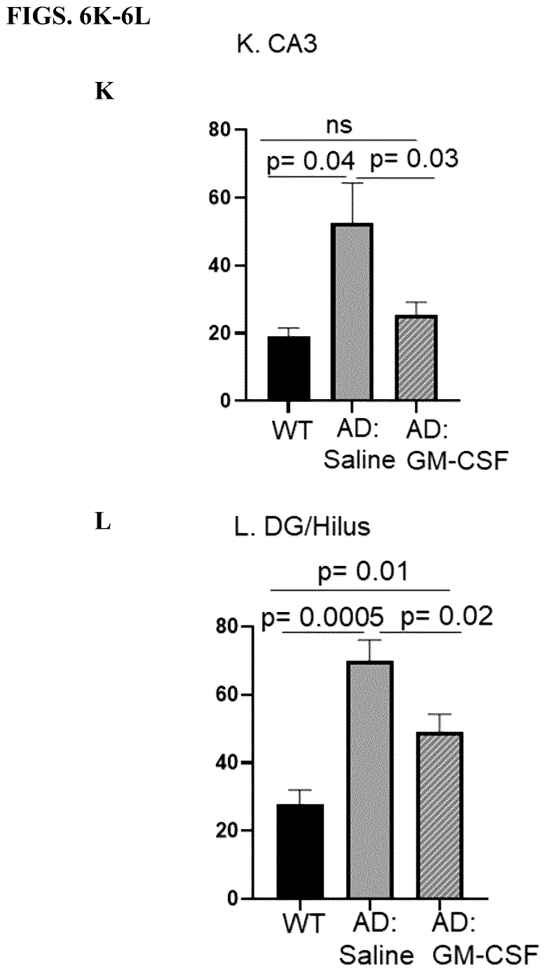

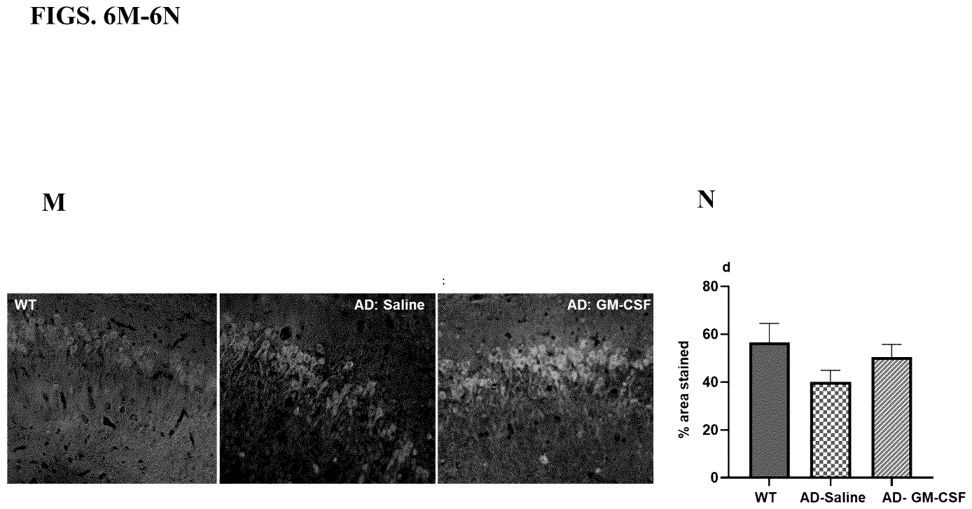

As illustrated in FIGS. 6A-6N, GM-CSF treatment of aged TgF344-AD rats significantly reduced the number of Caspase-3-positive cells in the CA1 region of the hippocampus compared to placebo-treated TgF344-AD rats (FIG. 6A-6C and FIG. 6J), and GM-CSF treatment reduced the number Caspase-3-positive cells in the CA3 region to nearly the same concentration as the age-matched F344 wild-type (WT) control rats (FIGS. 6D-6F and FIG. 6K). Further, levels of Caspase-3-positive cells in the dentate gyrus (DG) were also reduced following GM-CSF treatment in the same tested animals (FIGS. 6G-6I and FIG. 6L). Notably, most of the Caspase-3-positive cells in the CA1, CA3 and DG regions were neurons (approximately 95%) based on co-staining for the MAP2 neuronal marker (data not shown).

In another exemplary method as illustrated in FIGS. 6M-6N, brain sections were obtained from an animal model (male TgF344-AD rats (18-20 months)) treated with GM-CSF or placebo (saline injection) for five weeks that were also assessed for the level of neuronal integrity by immunohistochemical staining with ubiquitin C-terminal hydrolase-L1 (UCH-L1) (light gray, green in the original image), a marker for healthy neurons and with 4′,6-diamidino-2-phenylindole) (DAPI) ((dark spots of darker gray, blue in the original image), a nuclear counterstain blue-fluorescence dye, FIG. 6M). As illustrated in FIG. 6M, indirect immunofluorescence microscopy images demonstrated that neuronal expression of UCH-L1 in the hippocampus of untreated wild-type (WT, far left panel) rats, TgF344-AD rats treated with saline (AD: Saline, middle panel), and TgF344-AD rats treated with GM-CSF (AD-GM-CSF, right panel of FIG. 6M). For quantitative analyses, the percent area that stained positive for UCH-L1 (light gray, originally green in image) demonstrates a decrease of UCH-L1 in the hippocampus in the TgF344-AD rats compared to WT untreated rats (FIG. 6N). In contrast, the percent area staining positive for UCH-L1 where GM-CSF treatment was administered, demonstrates an increase of UCH-LI in the hippocampus of GM-CSF-treated TgF344-AD rats compared to saline-treated TgF344-AD rats (FIG. 6N) to nearly the same level as the WT rats (control, healthy rats) (n=4-7 rats/group). This observation of increased levels of UCH-LI in the brain tissue demonstrates that neuronal cell death can be ameliorated or restored to near normal levels when treated with GM-CSF in this AD model in support of ameliorating AD in human subjects.

### Example 7

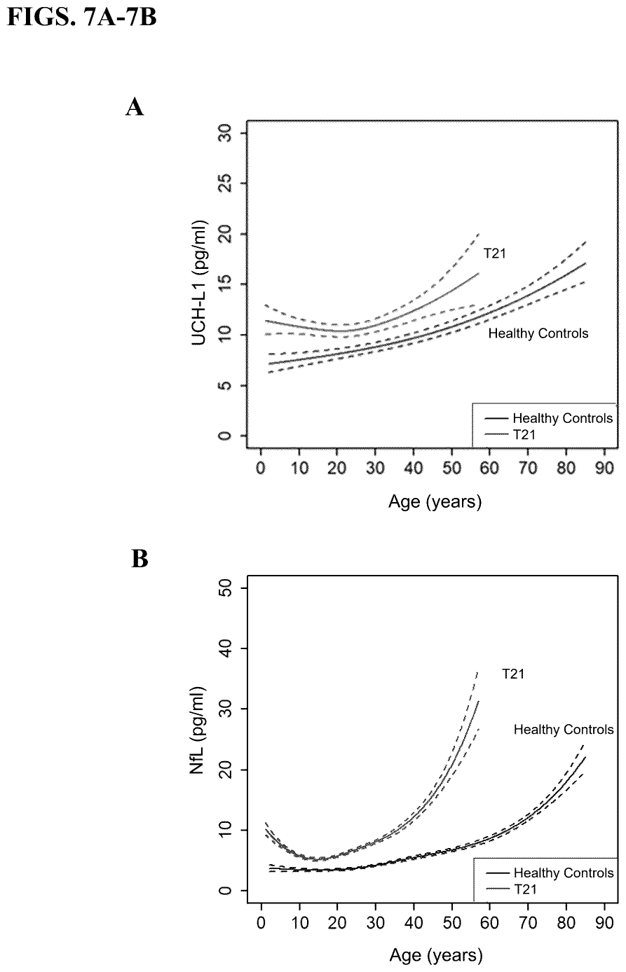

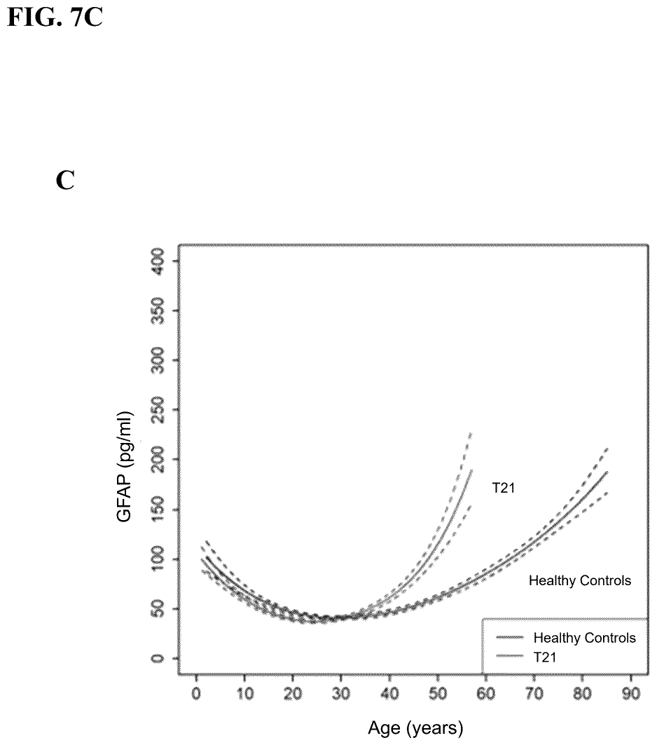

In another method, FIGS. 7A-7C illustrate some exemplary plots of concentrations of NfL, UCH-L1, and GFAP in plasma from patients with Down Syndrome compared to healthy controls (HC). The healthy adults carry a normal chromosome complement across the age range. These plasma biomarkers of neuronal death (UCH-L1; 7A) and damage (NfL; 7B) are higher at all ages of DS patients and rise exponentially faster than healthy controls, starting about age 25 and 15 respectively for these markers. A biomarker of inflammation, FIG. 7C (e.g., GFAP) is the same level for Trisomy 21 Down syndrome and normal healthy controls at young ages and then rises exponentially faster starting about age 35 in DS patients compared to healthy controls.

### Example 8

### Comparisons of Plasma Measures of Age-Associated Brain Neurodegeneration and Inflammation in MCI Due to AD, Mild-to-Moderate AD, and Healthy Controls

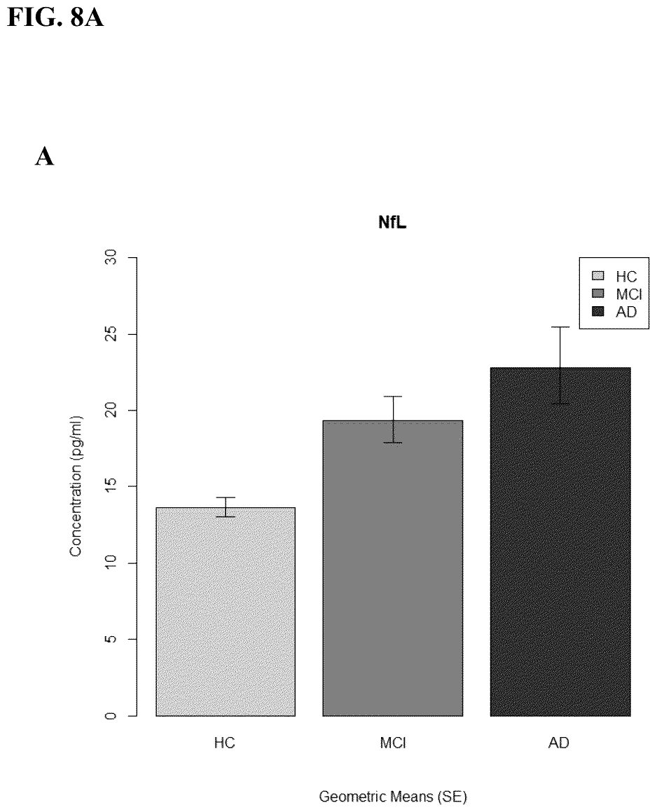

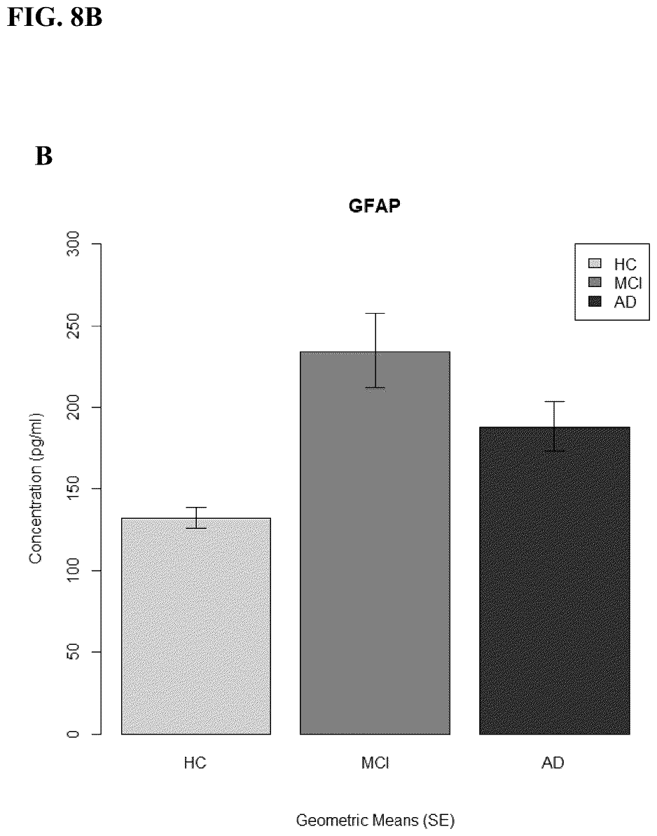

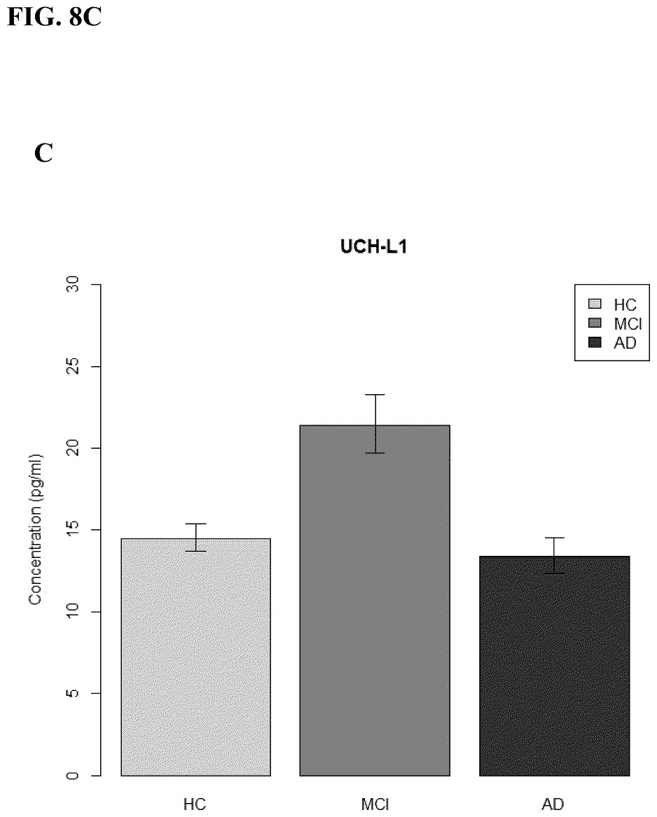

In another exemplary method, AD dementia and its precursor, MCI due to AD, are both strongly associated with age and are accompanied by neurodegeneration and astrogliosis in the brain. Having established full age curves for plasma markers of neurodegeneration (UCH-L1 and NfL) and astrogliosis/inflammation (GFAP), age-associated concentrations of NfL, GFAP, and UCH-L1 were compared in plasma samples from 32 participants with MCI due to AD from the Bio-AD study (MCI) and in plasma samples from 36 participants with mild-to-moderate AD at baseline (Baseline AD GM-CSF Study Pooled) (FIGS. 8A-8C). A diagnosis of MCI or mild to moderate AD was associated with higher overall levels of both NfL and GFAP compared to age-matched healthy control participants (FIGS. 8A-8B).

In other studies, it was observed that NfL concentrations were significantly higher in subjects with mild-to-moderate AD than in subjects with MCI, while there was little to no significant difference in GFAP concentrations between subjects with mild-to-moderate AD and subjects with MCI at 67.8 years, which is the mean age for the mild-to-moderate AD subjects (AD/HC estimate age 67.8: NfL: ratio estimate=1.852, 95% CI: (1.514, 2.265), p=2.201×10⁷; GFAP: ratio estimate=1.842, 95% CI: (1.539, 2.203), p=4.169×10⁹; MCI/HC estimate age 67.8: NfL: ratio estimate=1.362, 95% CI: (1.104, 1.682), p=0.0050; GFAP: ratio estimate=1.948, 95% CI: (1.473, 2.577), p=2.230×10⁵; AD/MCI estimate age 67.8: NfL: ratio estimate=1.359, 95% CI: (1.042, 1.772), p=0.0242; GFAP: ratio estimate =0.945, 95% CI: (0.699, 1.278), p=0.7091).

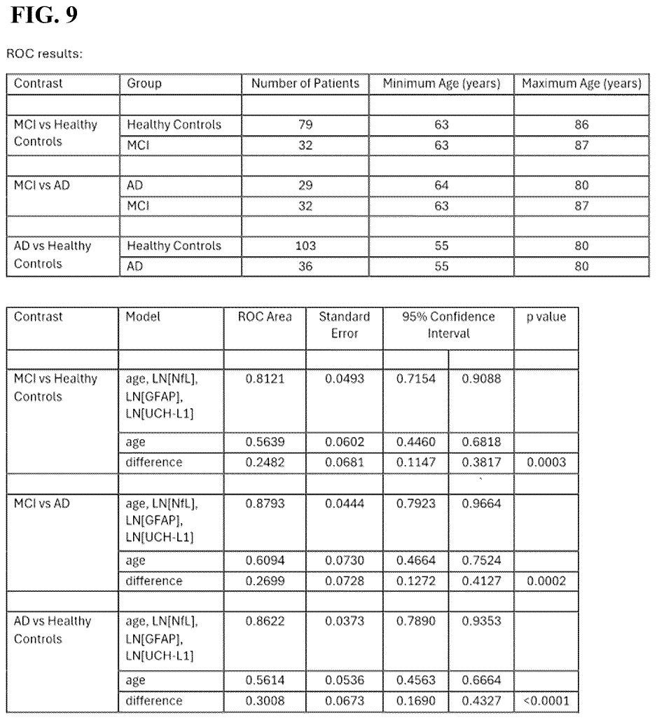

In another exemplary study, UCH-L1 levels in subjects with MCI due to AD demonstrated higher overall than the plasma UCH-L1 concentrations of the healthy control subjects at a mean age for the sargramostim/GM-CSF-treated mild-to-moderate AD participants, 67.8 years (ratio estimate=1.440, 95% CI: (1.101, 1.885), p=0.0088), whereas plasma UCH-L1 concentrations of the mild-to-moderate AD participants at the baseline visit prior to GM-CSF/sargramostim treatment are approximately the same as those of the healthy control participants (ratio estimate=0.967, 95% CI: (0.798, 1.171), p=0.7257) (FIG. 8C). UCH-L1 concentrations in MCI were higher than in mild-to-moderate AD at 67.8 years (ratio estimate=1.490, 95% CI: (1.133, 1.961), p=0.0052). UCH-L1 increased for participants with mild-to-moderate AD by an estimated 2.420% per year of age (95% CI: (0.783%, 4.083%), p=0.0054), As observed in these studies, because plasma concentrations of UCH-L1, NfL, and GFAP rise exponentially with increasing age and demonstrate differences between healthy controls (HC), MCI, and/or AD participants, they can serve as a single or combination novel biomarker assays to distinguish these groups in order to assess optimal diagnosis and/or intervention. FIG. 9 illustrates data, including the ‘Area Under the Curve; AUC’ from the ROC curves for NC (normal controls, healthy controls) vs MCI, NC vs AD and MCI vs AD for a combination of concentrations of UCH-L1, NfL, and GFAP in plasma samples while considering age, compared to the ROC curves for age alone. These results clearly demonstrate that the combination of these three biomarkers allows NC subjects to be distinguished from MCI subjects and from AD subjects; and MCI subjects to be distinguished from AD subjects. The ability to distinguish NC (normal control subject) from MCI subjects is critical for predicting risk of AD and where other biomarkers indicators have been unable to make this distinction, even in combination. Therefore, these procedures provide an important novel distinguishing assay to more accurately distinguish a subject's condition and potential intervention to reduce or inhibit progression of these health conditions.

As disclosed herein, identification of biomarkers of MCI and AD and one from the other is an area of active investigation, and these studies add an important biomarker (e.g., UCH-L1) for improving diagnosis and need for intervention. As provided herein, combining detection of concentrations of UCH-L1, NfL and GFAP, while taking age into consideration allowed discrimination between normal control (NC), MCI and AD in a subject. These results using plasma samples are at least comparable to, or better than those for many other biomarkers. For example, in a recent study using detection of pTau181 for a very difficult discrimination, between normal control (NC) vs MCI, detection of pTau181 was less effective than the combined detection of UCH-L1, NfL, and GFAP in a sample. One advantage of this combination biomarker based on UCH-L1, NfL, and GFAP together is that it directly measures the effect of disease on key brain cells, neurons and astrocytes, rather than relying on plasma proteins derived from intracellular or extracellular pathology or multiple proteins less directly linked to degeneration.

### Experimental Design

Participants: healthy control plasma samples were analyzed from three different studies. Healthy control participants (n=317; HTP, n=103; age range: 2-61 years; 54% female, (n=69; age range: 53-83; 70% female), or the multiple sclerosis (MS) healthy controls biomarker study (termed Nair) (n=145; age range: 16-86; 64% female). HTP is focused on studying biomarkers and clinical features of people with Down syndrome (DS) and includes typical control participants without DS, and the CUACC Bio-AD study is focused on studying the effect of inflammation on the development of AD. The Nair MS biomarker study is investigating biomarkers associated with MS and includes healthy control participants. Together, these three healthy control cohorts span ages 2-85. Using three community-dwelling healthy control cohorts that are diverse and heterogenous with a wide range of ages adds confidence to the cross-sectional measures of the plasma biomarkers.

Participants assessed as having mild cognitive impairment (MCI) due to AD (n=45) were diagnosed based on an interdisciplinary consensus conference with review of cognitive testing, neurological examination, clinical dementia rating scale (CDR), and brain MRI. Participants with mild-to-moderate AD were from a study, Phase II, double-blind, randomized, placebo-controlled trial with recombinant human GM-CSF. Available plasma samples taken at the baseline visit prior to treatment with sargramostim/GM-CSF or placebo (n=36), as well as available plasma samples taken at the end of three weeks of treatment with either GM-CSF (n=18) or placebo (n=18) were obtained. Some plasma samples were unavailable for measurements of NfL and GFAP concentrations.

Measurement of plasma biomarker concentrations: Concentrations of UCH-L1, GFAP, and NfL in plasma samples were assessed in healthy control participants and in participants with MCI due to AD using published methods and the Quanterix single molecule array, or SIMOA®, SR-X Analyzer system and the Neuro-4-Plex B kits. Concentrations of UCH-L1, GFAP, and NfL in the samples from mild-to-moderate AD participants in the sargramostim/GM-CSF AD trial were similarly determined.

### Statistical Analysis

Data were analyzed using mixed model regression, with unstructured error covariance on repeated measures, for the effects of biomarkers, disease status, and treatment on the logarithmic transforms of the plasma biomarkers of UCH-L1, GFAP, and NfL. Although data from healthy control participants and participants with MCI due to AD were cross-sectional, the framework of mixed model regression could still be adapted, as regression with independent data is merely a simplified version of regression with correlated data. Healthy control, MCI due to AD, mild-to-moderate AD at baseline, mild-to-moderate AD treated with placebo/saline, and mild-to-moderate AD treated with sargramostim/GM-CSF were allowed different covariance matrices. Because the biomarker data was measured using 2 or 3 replicates, all of the observations were used, instead of averaging replicates and weighting. The age effects were modeled as log-linear, with separate age slopes and intercepts for healthy control, MCI due to AD, and mild-to-moderate AD. The intercept for mild-to-moderate AD depended on the treatment x study time, but a common age slope was assumed for all mild-to-moderate AD participants, and independent of treatment effects. The log linearity assumption was checked graphically, and by comparing to spline fits 42. Interactions with sex were also considered. Linear combinations of model parameters were estimated, along with 95% confidence intervals, back transformed, and tested. Histograms of the Coefficient of Variation among replicates found some biomarker measures obtained with the SIMOA®, SR-X Analyzer system, particularly UCH-L1, for example, in analyses of the effects of TBI, can have Coefficients of Variance much higher than 20%, indicating that the variance is an inherent feature of the measure and not due, for example, to unreliable outliers. For example, a histogram of the UCH-L1 coefficient of variance of the healthy controls is demonstrated below:

For cross-sectional comparisons between large cohorts, the average or log average of individual participant's replicate measures is often used because measures of UCH-L1 often show high variance. UCH-L1 levels were FDA approved as part of a measure of brain damage after TBI. Because some of these cohorts had two replicates for each participant (healthy controls and participants with MCI due to AD from the Bio-AD) and some (healthy controls from the HTP, GM-CSF trial participants with mild-to-moderate AD, and healthy controls from the MS study) had mostly three replicates for each participant, and some of the biomarkers, especially UCH-L1, have high variability among replicates, it was decided in the modeling to use each replicate as a separate measure in the main analysis, rather than using the averages of the individual participants' replicate measures. The variation among replicates was modeled by introducing an additional noise term into the model. A common variance for the replicate noise term was assumed across all treatments because of software option limitations. Model predicted values of the response, along with pointwise standard errors, were plotted. All data calculations are available upon request. As a test of confidence in the results, the averages of the participants' replicate measures for the biomarkers were also modelled, weighted by the number of replicates, and the conclusions of age-associated exponential increases in the three biomarkers, UCH-L1, NfL, and GFAP, and the effect of GM-CSF treatment on reducing UCH-L1 levels were obtained. The overall results and conclusions were the same. Univariate statistical significance was set at alpha=0.05, two-sided, for all tests unless otherwise stated. Statistics were computed using SAS 9.4 and R 4.1.3.

1. A method for diagnosing a healthy subject with at least one of declining neuronal cell integrity and increasing neuronal cell damage, the method comprising:
(a) measuring a concentration of ubiquitin C-terminal hydrolase L1 (UCH-LI) in a first sample obtained from the healthy subject; and
(b) diagnosing the healthy subject with the at least one of declining neuronal cell integrity and increasing neuronal cell damage if the concentration of UCH-L1 in the first sample exceeds a concentration of UCH-L1 in a standard population of similar, same-aged, or younger healthy subjects to a tested healthy subject.

2. The method according to any one of claim 1, wherein the subject is diagnosed with at least one of declining neuronal cell integrity and increasing neuronal cell damage if the concentration of UCH-L1 in the first sample exceeds the concentration of UCH-L1 in the standard population of similar or same-aged healthy subjects by at least one, at least two, or at least three standard deviations of the standard population of similar, same-aged or younger healthy subject UCH-L1 concentrations.

3-4. (canceled)

5. The method according to claim 1, further comprising measuring a concentration of at least one of neurofilament light chain protein 1 (NfL) and glial fibrillary acidic protein (GFAP) in the first sample obtained from the healthy subject and comparing the concentration of the at least one of NfL and GFAP in the first sample to concentration of at least one of NfL and GFAP in a sample from a similar, same-aged or younger healthy subjects or standard population of similar or same-aged healthy subjects, and the healthy subject is diagnosed with a disorder if the concentrations of NfL and/or GFAP in the healthy subject exceeds concentrations in the similar or same-aged healthy subjects or standard population of similar, same-aged or younger healthy subjects.

6. The method according to claim 5, wherein measuring the concentration of at least one of neurofilament light chain protein 1 (NfL) and glial fibrillary acidic protein (GFAP) in the first sample obtained from the healthy subject comprises measuring the concentration of both NfL and GFAP in the first sample obtained from the healthy subject and comparing the concentration of the NfL and the GFAP in the first sample from the healthy subject to concentration of NfL and GFAP in the sample from the similar, same-aged or younger healthy subjects or the standard population of similar, same-aged or younger healthy subjects.

7. The method according to claim 1, wherein the first sample obtained from the subject comprises at least one of a blood and plasma sample.

8. (canceled)

9. The method according to claim 1, further comprising administering a treatment to the subject to treat at least one of the declining neuronal cell integrity and increasing neuronal cell damage.

10-12. (canceled)

13. A method of treating at least one of declining neuronal cell integrity and increasing neuronal cell damage in a subject, the method comprising
(a) measuring a concentration of UCH-L1 in a plasma sample obtained from the subject,
(b) comparing the concentration of UCH-L1 in the plasma sample with an average baseline concentration obtained from a standard healthy population of the same or similar age to the subject or younger than the subject,
(c) administering a treatment based on an elevated concentration level of UCH-L1 concentration in the plasma sample obtained from the subject compared to the average baseline concentration from the standard healthy population and treating the subject to reduce the at least one of declining neuronal cell integrity and increasing neuronal cell damage in the subject.

14. (canceled)

15. The method according to claim 13, further comprising: (d) measuring a concentration of UCH-L1 in at least a second plasma sample obtained from the subject after administration of the treatment in (c).

16. The method according to claim 15, further comprising, adjusting the treatment in (c) based on concentration of UCH-L1 in the at least a second plasma sample measured in (d) compared to the concentrations of UCH-L1 in the first plasma sample measured in (a); wherein increased concentration of UCH-L1 in the at least a second plasma sample comprises adjusting the treatment in (c) by increasing at least one of treatment frequency, treatment level or adding a second treatment to a regimen.

17. The method according to claim 13, wherein administering a treatment comprises administering an agent that decreases concentration of UCH-L1 in the plasma of the subject.

18. The method according to claim 17, wherein the agent for reducing concentration of UCH-L1 in the plasma of the subject comprises granulocyte-macrophage colony-stimulating factor (GM-CSF); optionally wherein the GM-CSF is recombinant GM-CSF.

19. (canceled)

20. The method according to claim 18, wherein the GM-CSF comprises sargramostim.

21-31. (canceled)

32. A method for testing efficacy of a targeted therapeutic agent, process, or task for treating at least one of declining neuronal cell integrity, increasing neuronal cell damage and increasing glial inflammation in a subject, the method comprising:
(a) measuring a concentration of UCH-L1 in a first sample obtained from the subject,
(b) administering the therapeutic agent, process, or task or combination thereof to the subject, and
(c) measuring a concentration of UCH-L1 after a predetermined period in at least a second sample obtained from the subject after administration of the therapeutic agent, process, or task or combination thereof, wherein efficacy of the potential therapeutic agent, process, or task or combination thereof, for reversing or reducing at least one of declining neuronal cell integrity, increasing neuronal cell damage and increasing glial inflammation is determined by a reduction in concentration of UCH-L1 in the at least a second sample compared to the first sample from the subject.

33. The method according to claim 32, wherein at least one of the first sample and the at least a second sample are each a plasma sample.

34-37. (canceled)

38. The method according to claim 13, wherein the subject is at risk of developing at least one of Alzheimer's Disease (AD), Parkinson's Disease, Down syndrome or Frontotemporal Dementia or other neurodegenerative brain disorder.

39. The method according to claim 1, wherein the subject is 5 years old or less.

40-46. (canceled)

47. The method according to claim 1, wherein the subject has a chromosomal abnormality related to a brain condition or cognition.

48. The method according to claim 32, wherein the process or task comprises at least one of an inhibitor of apoptosis or senolysis in neurons; an inhibitor of astrocyte activation that leads to at least one of lower GFAP expression and lower GFAP release into the plasma; an inhibitor or antagonist of receptors on astrocytes for signaling molecules; an inhibitor of microglial activation that results in at least one of lower expression and lower release of activating molecules, monoclonal antibodies against interleukin-1 (IL-1), interleukin-6 (IL-6), or other signaling molecules that activate astrocytes to make GFAP, and combinations thereof.

49. The method according to claim 48, wherein the inhibitor comprises an siRNA or a virus expressing siRNA.

50. The method according to claim 32, further comprising measuring concentration of at least one of neurofilament light chain protein 1 (NfL) and glial fibrillary acidic protein (GFAP) in the first sample and the at least a second sample from the subject and comparing concentration of the at least one of NfL and GFAP in the first sample compared to the second sample to assess efficacy of the targeted therapeutic agent, process, or task, wherein a lower concentration of at least one of NfL and GFAP in the at least a second sample indicates the targeted therapeutic agent, process, or task is treating the at least one of declining neuronal cell integrity, increasing neuronal cell damage, and increasing glial inflammation in the subject.

51-56. (canceled)
